(19) United States
(12) Patent Application Publication
(10) Pub. No.: US 2024/0393308 A1
(43) Pub. Date: Nov. 28, 2024

(54) DETECTION OF AIRBORNE CONTAMINANTS IN AN ENCLOSABLE ENVIRONMENT

(71) Applicant: Blue Core Technologies, LLC, Yellow Springs, OH (US)

(72) Inventors: Jason BAILEY, Plano, TX (US); Kyle BAILEY, Plano, TX (US); Keri Lee LESTAGE, Chicago, IL (US); Thomas Andrew WESTON, Winchester (GB)

(21) Appl. No.: 18/669,734

(22) Filed: May 21, 2024

Related U.S. Application Data
(60) Provisional application No. 63/468,186, filed on May 22, 2023.

(51) Int. Cl. G01N 33/00

(52) U.S. Cl. CPC G01N 33/0063; G01N 33/007; G01N 33/0075

(57) ABSTRACT

An apparatus, system, and method for determining a source of contaminants in air. The apparatus includes a plurality of sensors configured to generate raw sampling data of the air. The raw sampling data can describe at least two characteristics of the contaminants in the air. The apparatus also includes memory storing instructions and a processor communicatively coupled to the set of sensors and the memory, the processor configured to execute the instructions to cause the apparatus to identify one or more occurrences of a contamination event based on a comparison of the a characteristic and a detection threshold for the first characteristic of the contaminants in the air; and for each of the one or more occurrences of the contamination event, generate a data entry including a second characteristic. The second characteristic is usable to determine the source of the contaminants in the air.

### CROSS-REFERENCE TO RELATED APPLICATIONS

This application claims priority to U.S. Provisional Patent Application No. 63/468,186, filed May 22, 2023, which is incorporated herein by reference in its entirety for all purposes.

### TECHNICAL FIELD

Novel aspects of the present disclosure relate to the field of detection of contaminants, and more particularly, the detection of airborne contaminants in an enclosable environment.

### BACKGROUND

Significant amounts of revenue can be generated by the rental of property. Examples of rental property can include vehicles, houses, or rooms within buildings, e.g., hotel rooms or conference rooms. When the rental property is relinquished, it often needs to be cleaned before it can be placed back into inventory and relisted for rental. The use of certain substances in a rental property which generate airborne contaminants, like tobacco smoke, vaping vapors, and other illegal or illicit substances, can introduce contaminants that are difficult, if not impossible to remediate. For example, carpets, bedding, and upholstery exposed to cigarette smoke over extended periods of time may require complete replacement rather than cleaning. Thus, rental agreements often include provisions identifying prohibited substances and specify fines for violating those terms of the rental agreement.

The detection of one or more contaminants is generally performed after the rental property has been relinquished or returned. Oftentimes, the detection of the contaminants requires an inspector to enter an enclosed environment and attempt to detect evidence of prohibited substances by looking for visual clues or by trying to detect the odor of the prohibited substance in a subjective “sniff test”. Some inspectors might not be as sensitive to smells. Sometimes, temporary odor-masking substances may be deployed to mask the smell of the prohibited substances during the inspection period. Some inspectors may decline to actually inspect the interior of a rental property for fear of exposure to viral contaminants, like the SARS-CoV-2 virus responsible for the recent worldwide pandemic because the act of detecting the odor of prohibited substances would necessarily expose them to viral contaminants.

The existing procedure for inspecting returned or relinquished rental properties is time consuming and can expose the inspectors to airborne contaminants that could be injurious to health and promote the spread of pandemic-causing viruses. In addition, since the post-rental inspections are often subjective, such as the sniff test, the results may be contested by the renter. An improved system and method for objectively inspecting rental properties, and real-time detection data might resolve some of these issues.

In addition, there is also a market for detection of airborne contaminants in areas that are not within rental properties. Accordingly, the technology presented herein finds wide application in areas that may be subject to airborne contamination and that provide an opportunity for placement of the technology in that area to detect the contamination.

### SUMMARY OF THE INVENTION

Novel aspects of the present disclosure are directed to an apparatus for determining the nature or source of contaminants in air in an environment.

In an exemplary embodiment, the apparatus includes a plurality of sensors configured to generate raw sampling data of the air. In this embodiment, the raw sampling data can describe at least two characteristics of the contaminants in the air. The apparatus also includes memory storing instructions and a processor communicatively coupled to the set of sensors and the memory, the processor configured to execute the instructions to cause the apparatus to identify one or more occurrences of a contamination event based on a comparison of a first characteristic and a detection threshold for the first characteristic of the contaminants in the air; and for each of the one or more occurrences of the contamination event, generate a data entry including a second characteristic. The second characteristic is usable to determine the source of the contaminants in the air.

Novel aspects of the present disclosure are also directed to a method for determining a source of contaminants in air. In an exemplary embodiment, the method includes the steps of inducing air flow through a flow path contained within a housing; generating raw sampling data from the air flowing through the flow path, which can describe at least two characteristics of contaminants in the air; establishing a detection threshold for a first characteristic of the contaminants in the air; identifying one or more occurrences of a contamination event based on a comparison of the first characteristic and the detection threshold; and for each of the one or more occurrences of the contamination event, generating a data entry including the second characteristic. The second characteristic is usable to determine the source of the contaminants in the air.

Novel aspects of the present disclosure are also directed a system for determining a source of contaminants in air. In an exemplary embodiment, the system includes an apparatus mounted within an at least partially enclosed environment and configured to: generate raw sampling data of the air, which can describe at least two characteristics of the contaminants in the air; identify one or more occurrences of a contamination event based on a comparison of a first characteristic and a detection threshold for the first characteristic of the contaminants in the air; and for each of the one or more occurrences of the contamination event, generate a data entry including a second characteristic of the contaminants in the air. The system also includes a remote computing device located externally from the at least partially enclosed environment and configured to receive the data entry for each of the one or more occurrences of the contamination event. The apparatus or the remote computing device can determine the source of the contaminants in the air from the second characteristic.

The foregoing is a summary of exemplary embodiments and is not a detailed description of each aspect of the technology presented. Other aspects, embodiments and features of the invention will become apparent from the following detailed description of the invention when considered in conjunction with the accompanying figures.

### BRIEF DESCRIPTION OF THE FIGURES

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

FIG. 1 is a schematic diagram depicting a system for determining a source of contaminants in an enclosable environment according to an illustrative embodiment;

FIG. 2A is schematic diagram of a sensing apparatus for sampling air within an enclosable environment according to an illustrative embodiment;

FIG. 2B is a simplified block diagram of a sensing apparatus for sampling air within an enclosable environment according to an illustrative embodiment;

FIG. 2C is another schematic diagram of a sensing apparatus for sampling air within an enclosable environment according to an illustrative embodiment;

FIG. 3 is a block diagram of a mobile computing device for use in the detection and remediation of airborne contaminants according to an illustrative embodiment;

FIG. 4 is a schematic diagram of a server for use in the detection and remediation of airborne contaminants according to an illustrative embodiment;

FIGS. 5A, 5B, 5C, and 5D depict exemplary user interfaces provided on a mobile computing device for facilitating the detection and remediation of airborne contaminants in an enclosable environment according to an illustrative embodiment;

FIGS. 6A, 6B, 6C, 6D, 6E, and 6F depict exemplary user interfaces provided on a mobile computing device for facilitating the detection and remediation of airborne contaminants in an enclosable environment according to another illustrative embodiment;

FIG. 7 depicts an exemplary user interface for viewing raw sampling data according to an illustrative embodiment;

FIGS. 8A-8D depict raw sampling data captured in an enclosable environment over a two-hour period according to an illustrative embodiment;

FIGS. 9A-9D depict raw sampling data captured in an enclosable environment over a four-minute period according to an illustrative embodiment;

FIGS. 10-17 are various views of the sensing apparatus according to an illustrative embodiment;

FIGS. 18A and 18B are perspective views of the sensing apparatus according to an illustrative embodiment, but with the housing cover disengaged from its base;

FIG. 19 is a perspective view of a magnetic key for use with the sensing apparatus according to an illustrative embodiment; and

FIG. 20 is a flowchart of a process for detecting a source of contaminants in the air according to an illustrative embodiment.

### DETAILED DESCRIPTION

Aspects of this disclosure recognize the need for a sensing apparatus that can objectively detect the presence of airborne contaminants in real time in an enclosable environment without the need for human or animal inspectors to be exposed to the airborne contaminants and eliminating subjective elements of human sensory testing. Remediation efforts can be arranged immediately and/or automatically in response to detecting a presence of the airborne contaminants within the enclosable environment. In the commercial context, the objective data provided by the detection methods may also facilitate issuing of fines based on violations of a rental agreement and make it unlikely for a renter and/or occupant of the rental property to successfully dispute the violations. Of course, the technology also finds application in non-rental contexts, for example in the home, or in personally owned cars, boats, and the like, which are subject to air contamination by, for example and without limitation, combustion products of tobacco and cannabis, vape products, and the like. Not only does the inventive technology provide detection and identification of the contaminant, but it also provides a time when the contamination event occurred, and if the technology is deployed in a mobile space, such as the cabin of a car, or other mobile space, it identifies the place where the contamination event occurred. The identification of the contaminant and time of the contamination event is commercially useful in, for example, without limitation, the hospitality industry where the hotel or AirBnB guest responsible for the contamination event can be identified objectively. The identification of the time and place of the contamination event is commercially useful, for example, without limitation, in the automobile rental industry where the renter at the time of the contamination event is known, and the place where the contamination event occurred, provides an objective basis for renter responsibility. Of course, it is readily apparent that there are residential applications and a wide range of commercial applications. Exemplary embodiments of the inventive technology are described herein, and claimed here below.

The exemplary system described in the disclosure that follows is directed to airborne contaminants. In the present context, an airborne contaminant is a presence in the air of an undesirable composition or particulate that may cause an undesirable odor, and/or that may pose health risks. Examples of such contaminants include the components of smoke that originates from the combustion of tobacco products, components of the combustion of Cannabis or vapors (volatile organic compounds aka “VOCs”) or particulates that may arise from “vaping” which has become increasingly prevalent. The detection device of the present invention may be modified to detect other substances, including other controlled or illicit substances. Embodiments of this disclosure can be used to detect the presence of biological contaminants, such as mold.

In the present context, an “enclosable environment” or “enclosable space” means any space that can be isolated from its surroundings. This isolation may be by closing doors, windows, a roof (e.g., of a convertible car) and any other means of ingress or egress to the space but does not necessarily include closing air vents which may be sources of the contamination. Non-limiting examples of sources of the contamination can include tobacco products, vaping products, Cannabis products, etc.

While not a subject of this technology, there are technologies available commercially to decontaminate areas found to be contaminated. These include, for example, the patented technology of Nu VinAir LLC that utilizes a “gaseous cleaning agent” is used to remediate contaminated enclosable spaces and surfaces in these spaces. Details of the patented Nu VinAir technology may be found, for example, in U.S. Pat. No. 9,446,742 (Reexam No. 90/019,043); U.S. Pat. No. 9,925,959 (Reexam No. 90/019,044); U.S. Pat. Nos. 11,420,599; 11,535,205; and U.S. patent application Ser. Nos. 17/989,454 and 17/989,454, which are incorporated by reference to the extent consistent with the disclosures herein.

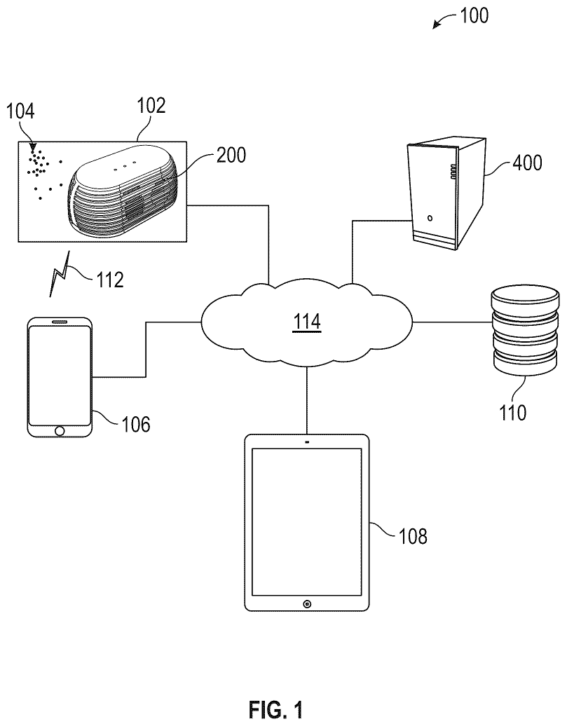

FIG. 1 is a system 100 for real-time detection and remediation of airborne contaminants in an enclosable environment according to an illustrative embodiment. Non-limiting examples of the enclosable environment 102 can include a room within a building, such as a room in a house or hotel, or the cabin of a vehicle, such as without limitation, a car cabin or a recreational vehicle, or a boat cabin, or an airplane cabin or lavatory.

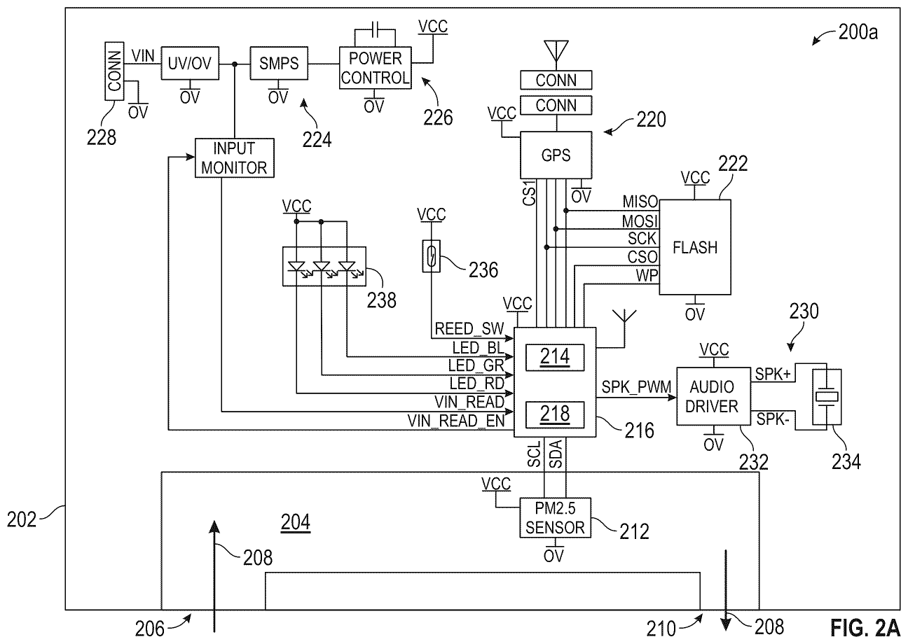

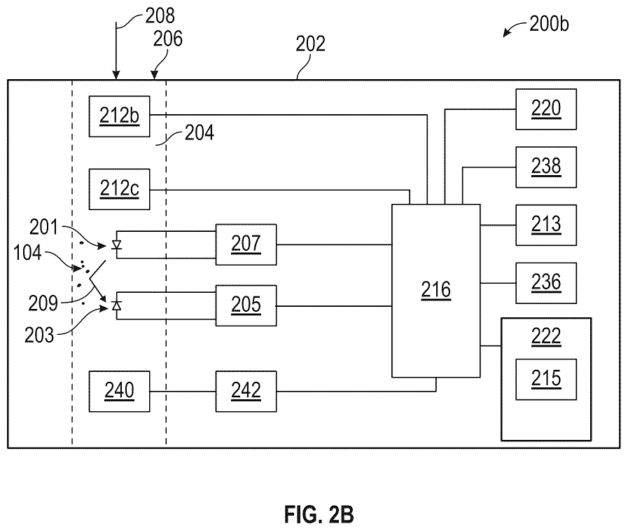

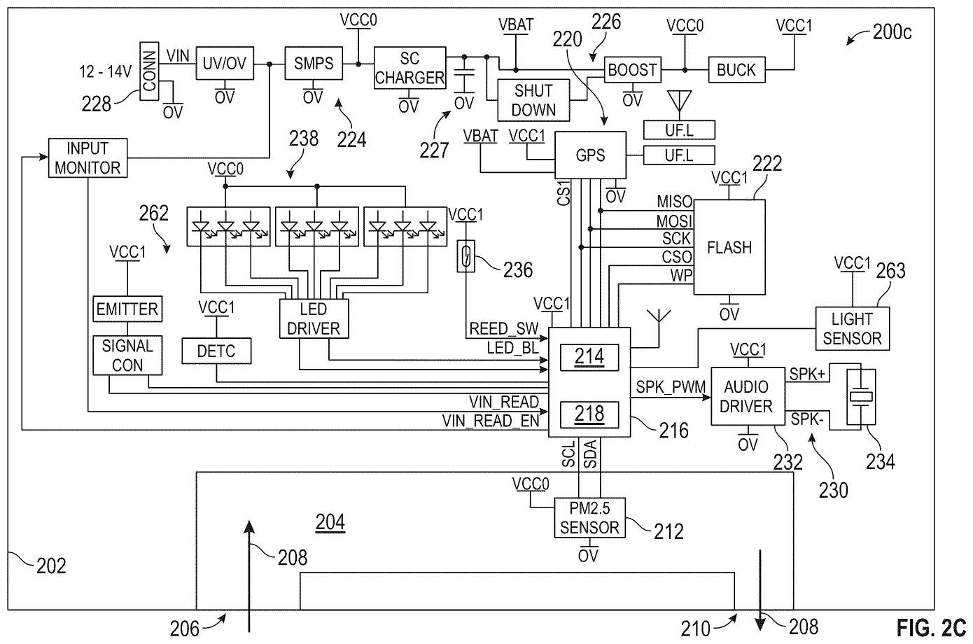

Generally, the system 100 includes a sensing apparatus 200 that samples air within the enclosable environment 102. Embodiments of sensing apparatus 200 are described in more detail in FIGS. 2A, 2B, and 2C that follows. For the sake of simplicity, the sensing apparatus 200a in FIG. 2A, the sensing apparatus 200b in FIG. 2B, and the sensing apparatus 200c in FIG. 2C can be referred to collectively as sensing apparatus 200.

Raw sampling data captured by the sensing apparatus 200 can be used to identify the one or more airborne contaminants 104, i.e., to determine the source of the one or more airborne contaminants. Once the source of the one or more contaminants have been identified, remediation of the enclosable environment 102 can be initiated. Remediation can include scheduling cleaning of the enclosable environment 102 and, in the event that the enclosable environment 102 is a rental property, the system 100 can update an inventory to indicate when the enclosable environment 102 will be ready for use post-remediation. Additionally, in the event that one or more airborne contaminants 104 include an illicit substance, an illegal substance, or an otherwise prohibited substance as defined by law and/or a corresponding rental/space occupation agreement, remediation can include determining whether presence of the substance violates the agreement to permit automatically assessing a fine. In addition to detecting the presence of one or more airborne contaminants, such as tobacco smoke, vaping vapors/particulates, and volatile organic compounds (“VOCs”), the sensing apparatus 200 can also detect fluctuations in ambient conditions consistent with the use of prohibited substances, such as changes in temperature and/or humidity within the space being monitored by the sensing device.

In some embodiments, the sensing apparatus 200 can also capture contextual data related to the raw sampling data. The contextual data can be used to provide additional context for the raw sampling data. Examples of contextual data might include, but are not limited to, a time and a location related to the sampling data, and/or an identifier associated with one or more renters or occupants of the enclosable environment 102. The identifier might be, for example, a unique identifier of a mobile phone, a voice sample, or photograph. An example of some raw sampling data and contextual data that can be captured by the sensing apparatus 200 and included in a data entry for use in determining the source of contaminants in air is shown in Table 1, below.

TABLE 1
Data Entry of Raw Sampling Data and Contextual Data

| DATA TYPE | VALUE |
|---|---|
| LATITUDE | 33.081111234578344 |
| LONGITUDE | −96.8235841085388 |
| PM1 | 4626.6 |
| PM2.5 | 6553.4 |
| PM4 | 6553.4 |
| PM10 | 6553.4 |
| HUMIDITY | 46 |
| TEMP | 24.1 |
| VOC | 160 |
| NOX | 1 |
| TIMESTAMP | 01/01/2024 8:16 |
| SPEED OVER | 0.25 |
| GROUND | |
| (SOG/MPH) | |
| COURSE OVER | 0 |
| GROUND | |
| (COG/ANGLE) | |

In Table 1, the raw sampling data includes particle mass concentration, e.g., PM1, PM2.5, PM4, and PM10, as well as humidity, temperature, and relative amounts of volatile organic compounds (VOCs) and nitrogen oxides (NOx). The contextual data includes location data, e.g., latitude and longitude, which can be used to determine where the air samples were taken, timestamp information, as well as movement-specific data, e.g., speed over ground and course over ground. The exemplary data values in Table 1 indicate a presence of VOCs in the air sample, which can be used to determine the use of tobacco products in the enclosable environment 102. As will be described in more detail below, the mass concentration exceeding a detection threshold indicates an occurrence of a contamination event that results in the generation of a data entry that includes raw sampling data and the contextual data. The magnitude of the VOC value can then be used to determine that the source of the contaminants in air is from a tobacco product. Vaping in the enclosable environment generates undetectable amounts of VOCs.

In some embodiments, the sensing apparatus 200 is configured to perform sampling on the air within the enclosable environment 102 and also analyze the raw sampling data to generate sampling results that identify the presence of the airborne contaminants 104 in the enclosable environment 102. The sensing may take place continuously or at prescribed intervals. The sensing apparatus 200 can then send the sampling results (and optionally the raw sampling data and the contextual data, if available) captured in real time to a remote computing device located externally to the enclosable environment 102, either proximate to or remote from the enclosable environment 102. The remote computing device can be a client device, such as mobile computing device 106 operated by a rental car agency, a hotel front desk, or any other client of a system provider, or mobile computing device 108 operated by the system provider. The remote computing device can also be a remote server, such as server 400. The sampling results, the raw sampling data, and/or the contextual data can be stored in one place or several places, for example, without limitation, in the local storage of the sensing apparatus 200, such as local storage 222, in the remote computing device or a network accessed storage device, such as network accessible storage 110. (For the sake of convenience, the raw sampling data, the sampling results, and/or the contextual data may be referred to simply as “sensing data”.) The sensing data can be used to provide an alert in real time to allow a responsible party to initiate remediation of the enclosable environment 102 and to manage associated business records, such as rental agreements and to take actions in accordance with these agreements.

In other embodiments, the sensing apparatus 200 is configured to only perform sampling of the air within the enclosable environment 102 and a remote computing device located externally to the enclosable environment 102 performs analysis of the raw sampling data and the contextual data, if any, to generate the sampling results. For example, mobile computing device 106 may generate the sampling results from the raw sampling data and contextual data, if any, provided by the sensing apparatus 200. Alternatively, mobile computing device 106 can transmit the raw sampling data and contextual data, if any, over a network in real time to the network accessible storage or to another computing device tasked with analyzing the raw sampling data to generate sampling results. The remote computing device generating the sampling results can also prompt initiation of remediation of the enclosable environment 102, if necessary, and determine compliance with an agreement, such as for example a rental agreement if the enclosable environment 102 is rental property. Alternatively, a first remote computing device may be configured to generate the sampling results and a second remote computing device may be configured to initiate and/or manage remediation of the enclosable environment 102, and/or determine compliance with a rental agreement if the enclosable environment 102 is rental property.

Sensing data may be stored locally on the sensing apparatus 200 and transmitted to the remote computing device when a communications link is available. The communications link can be any wired or wireless communications link. The communications link can allow the sensing apparatus 200 to communicate with one or more mobile computing devices 106, 108, and/or server 400, directly via device-to-device communications links, like communications link 112 or over network 114. For example, the communications link 112 can be a BLUETOOTH® connection that allows the sensing apparatus 200 to exchange data directly with mobile computing device 106 when the two devices are within range. The mobile computing device 106 may then send the data over network 114 using a cellular communications link. In another example, the communications link can be a WIFI® connection that allows the sensing apparatus 200 to send and receive data over network 114 in the presence of a hotspot.

In certain embodiments in which the sensing apparatus 200 includes a dedicated communications link permitting the sensing apparatus 200 to communication over network 114, such as a cellular connection, the sensing data can be transmitted to one or more remote computing device continuously, periodically, or intermittently, e.g., only when the sampling results indicate that the one or more airborne contaminants 104 has been detected within the enclosable environment 102. Although more convenient, the dedicated communications link may add to manufacturing costs, increase the form factor of the sensing apparatus 200 and use more power. Nonetheless, it may find application in certain circumstances, such as, but not limited to, an airplane lavatory, or a hotel room.

Some examples are provided to illustrate the exchange of sensing data in the various non-limiting use cases described above.

### Examples

Example 1. The sensing apparatus 200 captures raw sampling data from within the enclosable environment 102 and analyzes the raw sampling data to generate sampling results. The sensing apparatus 200 optionally captures contextual data. The sensing data is transmitted to remote computing device, such as mobile computing device 106. The remote computing device may be configured to initiate steps for remediation of the enclosable environment 102 based on receiving sampling results indicating a presence of one or more airborne contaminants 104. In the event that the enclosable environment 102 is a rental property, for example, the remote computing device determines compliance with an associated rental agreement and issues a fine, if appropriate. The remote computing device can also upload the sensing data to storage device 110 to permit access by one or more remote computing devices over network 114.

Example 2. The sensing apparatus 200 captures raw sampling data from within the enclosable environment 102 and analyzes the raw sampling data to generate sampling results. The sensing apparatus 200 optionally captures contextual data. The sensing data is transmitted to a remote computing device, such as mobile computing device 106. The remote computing device uploads the sensing data to a storage device 110 to permit access by one or more remote computing devices over network 114. For example, server 400 can access the sensing data from the storage device 110 for initiating remediation, determining compliance with an associated rental agreement, and issuing a fine, if appropriate. In some embodiments, the remote computing device receiving the sensing data from the sensing apparatus 200 may also transmit the sensing data directly or one or more remote computing devices, such as mobile computing device 108 and/or server 400.

Example 3. The sensing apparatus 200 captures raw sampling data from within the enclosable environment 102 and optionally captures contextual data. The raw sampling data and the contextual data, if any, are transmitted to remote computing device, such as mobile computing device 106. The remote computing device is capable of analyzing the raw sampling data to generate sampling results and can initiate remediation of the enclosable environment 102 based on the sampling results indicating a presence of one or more airborne contaminants 104. In the event that the enclosable environment 102 is a rental property, the remote computing device can determine compliance with an associated rental agreement and issue a fine, if appropriate. The remote computing device can also upload the sensing data to storage device 110 to permit access by one or more other remote computing devices over network 114.

Example 4. The sensing apparatus 200 captures raw sampling data from within the enclosable environment 102 and optionally captures contextual data. The raw sampling data and the contextual data, if any, are transmitted to remote computing device, such as mobile computing device 106. The remote computing device uploads the raw sampling data and the contextual data, if any, to a storage device 110 to permit access by one or more other remote computing devices over network 114. The other remote computing devices can access the raw sampling data and the contextual data, if any, to generate sampling results, initiate remediation of the enclosable environment 102, determine compliance with an associated rental agreement, and issue a fine, if appropriate. In some embodiments, the remote computing device receiving the sensing data from the sensing apparatus 200 may also transmit the sensing data directly or one or more remote computing devices, such as mobile computing device 108 and/or server 400.

Example 5. The sensing apparatus 200 captures raw sampling data from within the enclosable environment 102 and optionally captures contextual data. The sensing apparatus 200 analyzes the raw sampling data and generates sampling results and transmits the sensing results and the contextual data over network 114 in response to detecting the availability of a networked communications link. The sensing results and contextual data can be transmitted to storage device 110 and/or to another remote computing device, such as mobile computing device 106, 108, and/or server 400. One or more of the remote computing devices can initiate remediation, if necessary, and generate business records referencing the sampling results.

Example 6. The sensing apparatus 200 captures raw sampling data from within the enclosable environment 102 and optionally captures contextual data. The sensing apparatus 200 transmits the raw sampling data and the contextual data over network 114 in response to detecting the availability of a networked communications link. The raw sampling data and the contextual data can be transmitted to storage device 110 and/or to one or more other remote computing devices, such as mobile computing device 106, 108, and/or server 400. The one or more other remote computing devices can then generate the sensing results, initiate remediation, and generate business records referencing the sampling results.

When the enclosable environment 102 is rental property subject to a rental agreement and the sensing data does not indicate a presence of one or more airborne contaminants 104 within the enclosable environment 102, then the owner of the property (for example, without limitation, a rental car company or a hotel) does not need to carry out remediation. A remote computing device can update the inventory to include the availability of the enclosable environment (vehicle or room, under the example) for immediate use.

In the event that the sensing data does indicate a presence of the one or more airborne contaminants within the enclosable environment 102, the remote computing device can automatically initiate remediation or signal that remediation is needed generating a message that identifies the enclosable environment 102. The message may also identify the one or more airborne contaminants present in the enclosable environment 102, and an amount of the one or more airborne contaminants 104. The message can be sent to a mobile device operated by a person tasked with cleaning of the enclosable environment 102, such as an owner, property manager, third-party vendor, or renter.

FIG. 2A is schematic diagram of a sensing apparatus for sampling air within an enclosable environment according to an illustrative embodiment. The sensing apparatus 200a includes a housing 202 that defines a flow path 204 in fluid communication with the enclosable environment 102. The flow path 204 has an inlet 206 that receives air, represented by arrow 208, from the enclosable environment 102 and an outlet 210 that expels air 208 into the enclosable environment 102. A sensor 212a is exposed to the air 208 in the flow path 204 so that particulate matter suspended in the air 208 of the enclosable environment 102 can be detected by the sensor 212a as the air is conveyed through the flow path 204. The sensor 212a can use any currently existing (or later developed sensing technology), including infrared detection, beta attenuation mass monitoring, and laser diffraction, as long as it is configurable for the detection of the contaminants at issue. The data captured by sensor 212a may also referred to in the alternative as “raw sampling data.”

Although not shown in FIG. 2A, the sensing apparatus 200a can include a device, such as a fan, to induce air flow from the enclosable environment 102 into the flow path 204. In an exemplary embodiment, the sensor has an associated fan that continuously draws air from the environment to the sensor (or plurality of sensors). Alternatively, the sensing apparatus 200a can be placed in a discrete location that is not readily visible to occupants of the enclosed environment 102, but which can receive circulated air within enclosable environment 102. For example, without limitation, the flow path 204 can be fluidically connected to ducting within the enclosable environment 102 to capture circulating air. The ducting can be a circulation system of a home or a vehicle. In other embodiments the apparatus may be placed in an area or region to which air may be expected to flow. An example of such an area in a vehicular cabin might be, for example, the ledge adjacent to the rear window and behind a rear seat of a sedan, where cigarette smoke might migrate (or be induced by a fan associated with the sensors) in a moving car, typically with windows closed.

In some embodiments, the sensing apparatus 200a is capable of analyzing the raw sampling data to generate sensing results identifying the contaminant detected, or at least identifying characteristics that can be attributed to the presence of certain contaminants, such as the increase in VOCs, particle sizes, ratios of particle sizes, temperature, and/or humidity. The analysis can be performed by processor 214 that is communicatively coupled to the sensor 212a. The processor 214 can use any currently existing (or later developed) algorithms to analyze the raw sampling data to form sensing results. In this illustrative embodiment, the processor 214 is integrated in a microcontroller 216. The microcontroller 216 also includes a communications interface 218 that allows the sensing apparatus 200a to exchange data with remote computing devices. The communications interface 218 can provide wireless communications links using BLUETOOTH® or WIFI® protocols.

The processor 214 can also cause the sensing apparatus 200a to collect the contextual data that can be used to provide additional context for the raw sampling data and/or the sensing results. For example, the processor 214 can cause the GPS device 220 to gather location data, and optionally time data, continuously, periodically, or intermittently, e.g., only when the sensing apparatus 200a detects a presence of the one or more airborne contaminants 104 within the enclosable environment 102. Additionally or in the alternative, the processor 214 can capture time data continuously, periodically, or intermittently using an embedded clock or timekeeping circuit (not shown). Thus, in embodiments, where the processor 214 can also detect a presence of the one or more airborne contaminants 104 within the enclosable environment 102, the processor 214 can be configured to generate sampling results that include at least: an identity of the airborne contaminant and an amount of the airborne contaminant, along with an associated time stamp and a location stamp. In the event that sampling results are determined by a remote computing device, the contextual data provided to the remote computing device along with the raw sampling data can be used by the remote computing device to generate sampling results that include an identity of the airborne contaminant and an amount of the airborne contaminant, along with an associated time stamp and a location stamp.

Sensing data can be stored in local storage 222. In this illustrative embodiment, local storage 222 is flash memory, but in other embodiments other types of memory can be used in addition to or instead of flash memory.

Although not shown in the sensing apparatus 200a in FIG. 2A, general location data can be provided by non-GPS technologies. For example, if sensing apparatus 200a includes WIFI or cellular communications capability, processor 214 can generate approximate location data based on proximity to stationary WIFI-enabled devices with a known location, or triangulation with cell towers. Likewise, BLUETOOTH communications with stationary BLUETOOTH-enabled devices can also be used for establishing general location data.

Sensing apparatus 200a may be powered by power supply 224. In a first embodiment, the power supply 224 provides the sensing apparatus 200a with power from a local power supply, such as a (rechargeable) battery. In a second embodiment, the power supply 224 provides the sensing apparatus 200a with power from an external power supply, such as an electrical system of the enclosable environment 102. For example, if the enclosable environment 102 is a vehicle, then the power supply 224 can be connected directly to the vehicle battery or to a wiring harness electrically connected to the vehicle battery, which only provides power to the sensing apparatus 200a when the vehicle is running or in accessory mode. In a third embodiment, which is depicted in FIG. 2A, the power supply 224 can provide the sensing apparatus 200a with power from a local power supply 226 and also an external power supply via connectors 228, which can be connected to an electrical system of the enclosable environment 102. The local power supply 226 can be a rechargeable battery or a supercapacitor that can store excess electrical charge when the external power supply provides power to the sensing apparatus 200a through connectors 228.

In exemplary embodiments, the sensing apparatus 200a may include an audio subsystem 230 that can generate an audible signal when the sensing apparatus 200a is disconnected. The audible signal may dissuade occupants of an enclosable environment 102 from tampering with the sensing apparatus 200a. The audio subsystem 230 includes an audio driver 232 and speakers 234. The audio driver 232 provides a signal to drive the speakers 234 in response to a “triggering condition.” The triggering condition might be for example, the removal of the apparatus 200a from its mounted position. For example, the sensing apparatus 200a can be mounted on a magnetic base (not shown). The magnetic field from the magnetic base can cause the reed switch 236 to adopt a first configuration, e.g., either open or closed. Removal of the sensing apparatus 200a from the magnetic base can cause the reed switch 236 to adopt a second configuration that is different from the first condition, e.g., from open to closed or closed to open. The second configuration can cause the audio driver 232 to send a signal to the speaker 234 which generates the audible signal. In some embodiments, the audio driver 232 can be electrically coupled to speakers present in the enclosable environment, e.g., the car speakers, to cause the car speakers to generate the audible signal.

In a non-limiting embodiment, the sensing apparatus 200a can also include Input/Output (I/O) 238, which is an interface that conveys information to a user. In this illustrative embodiment, I/O 238 is a series of multi-colored LEDs that can illuminate a certain color or pattern to give an indication of the operational status of the sensing apparatus 200a, e.g., data transfer, sampling, malfunction, standby, etc.

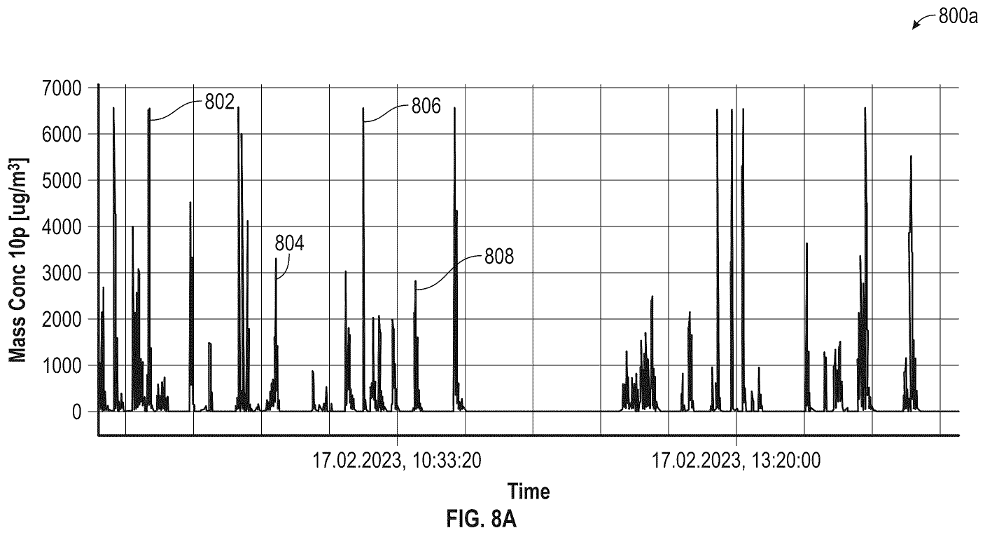

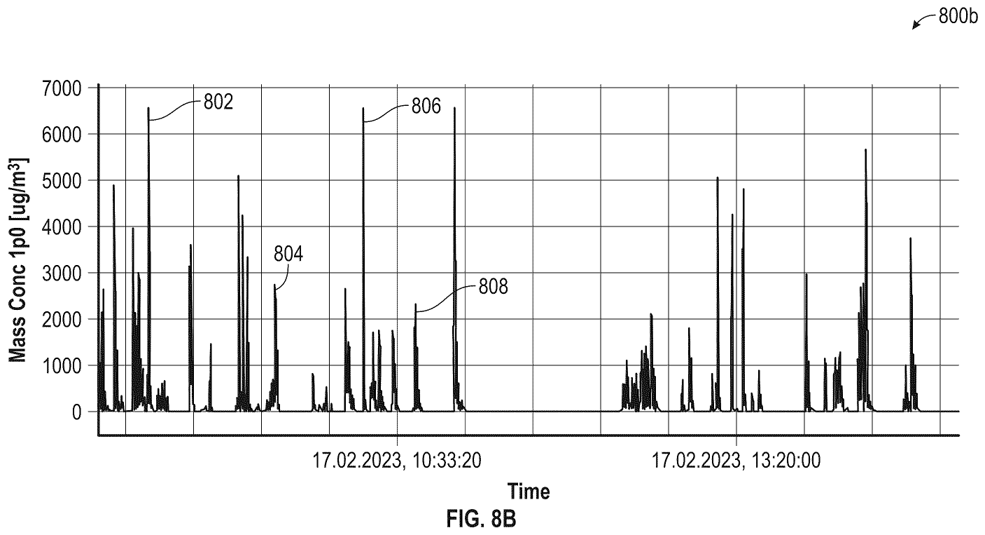

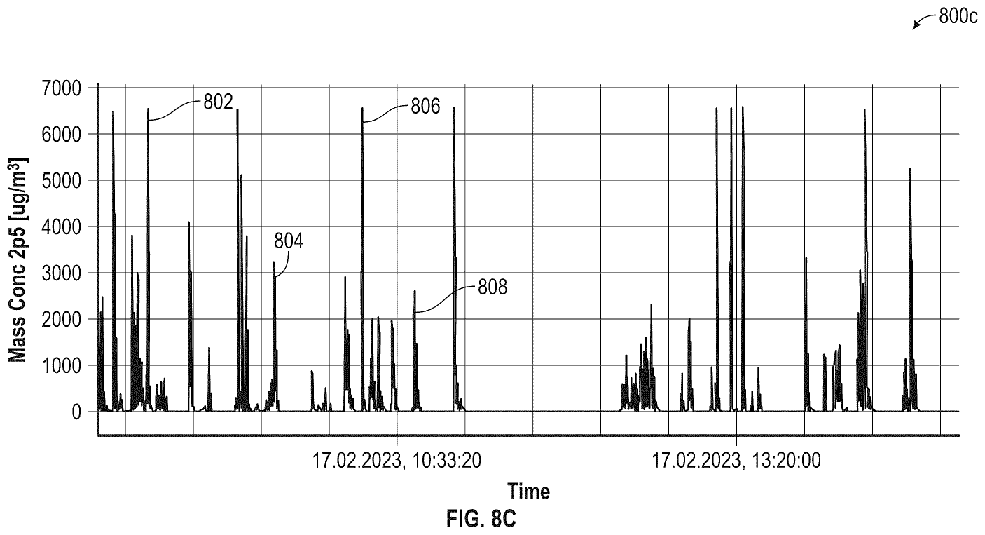

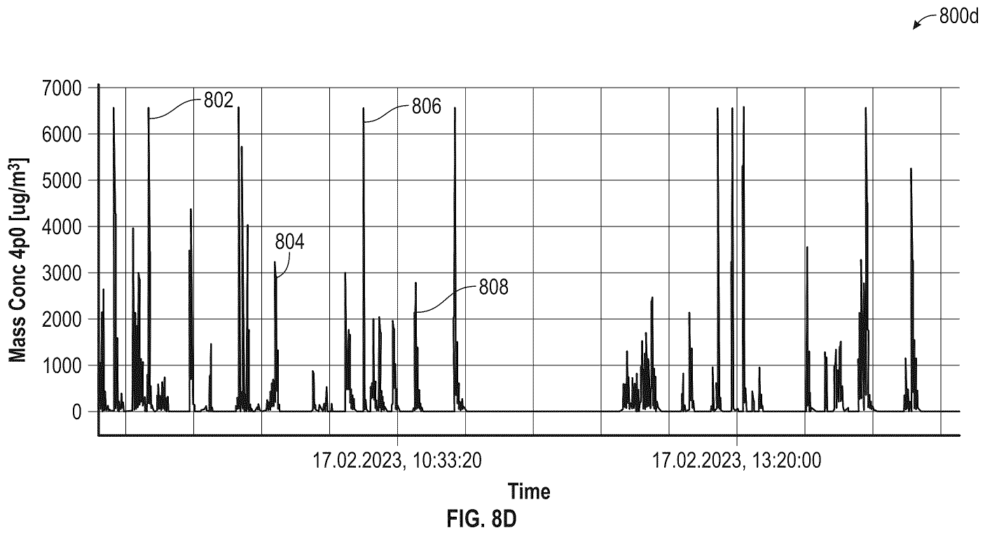

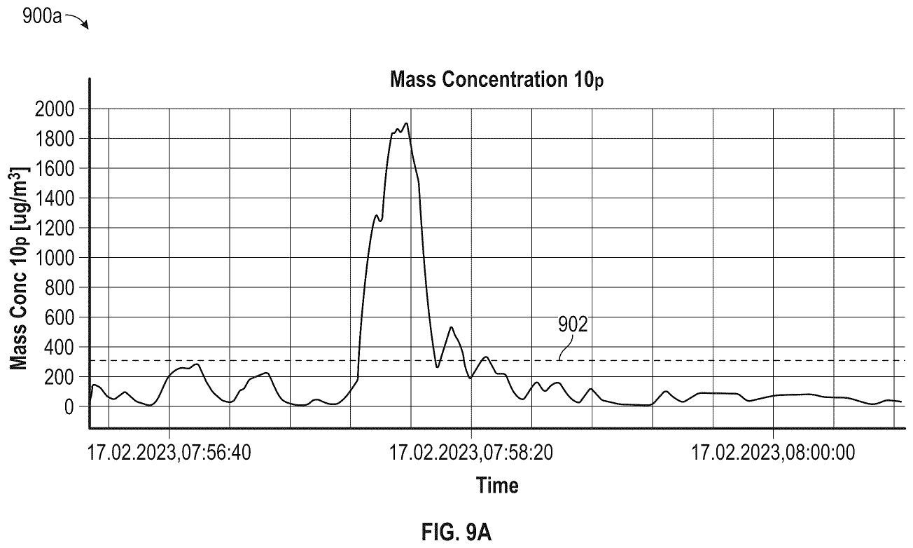

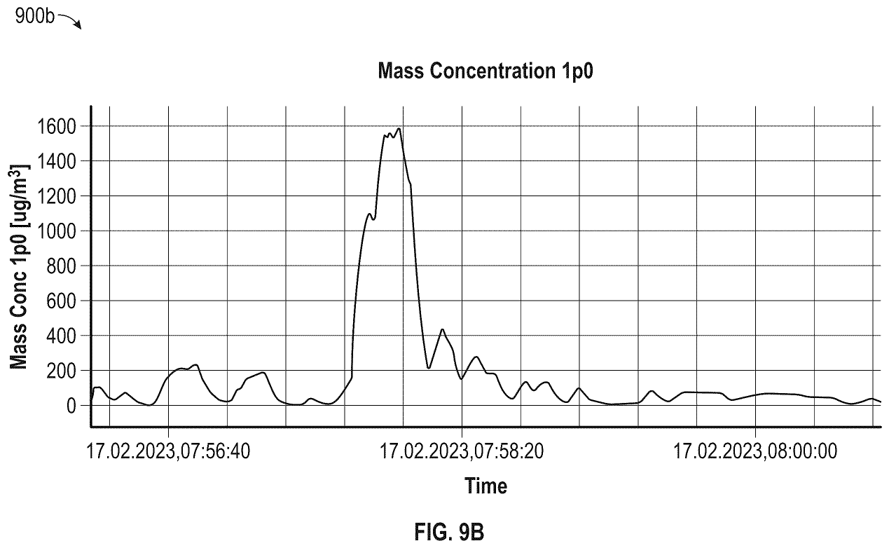

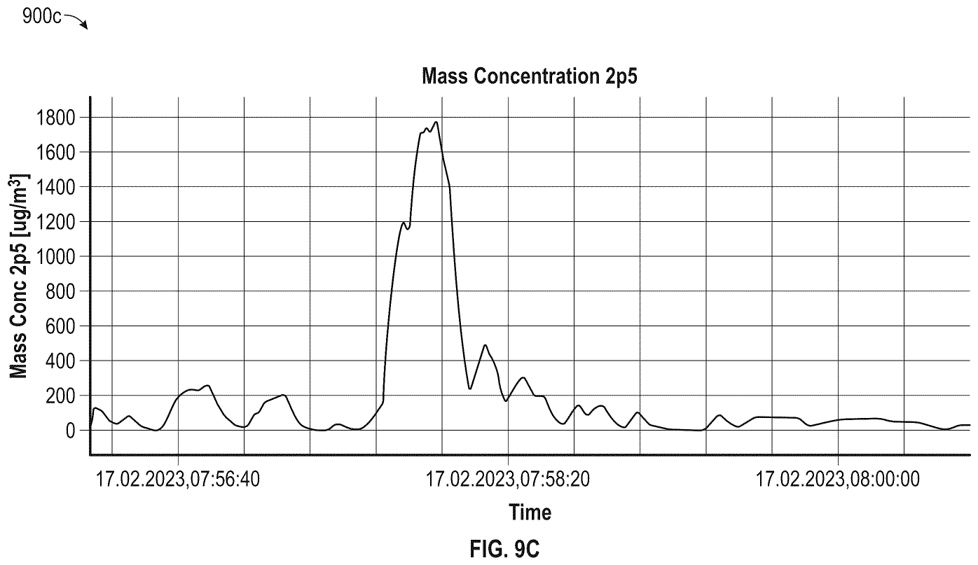

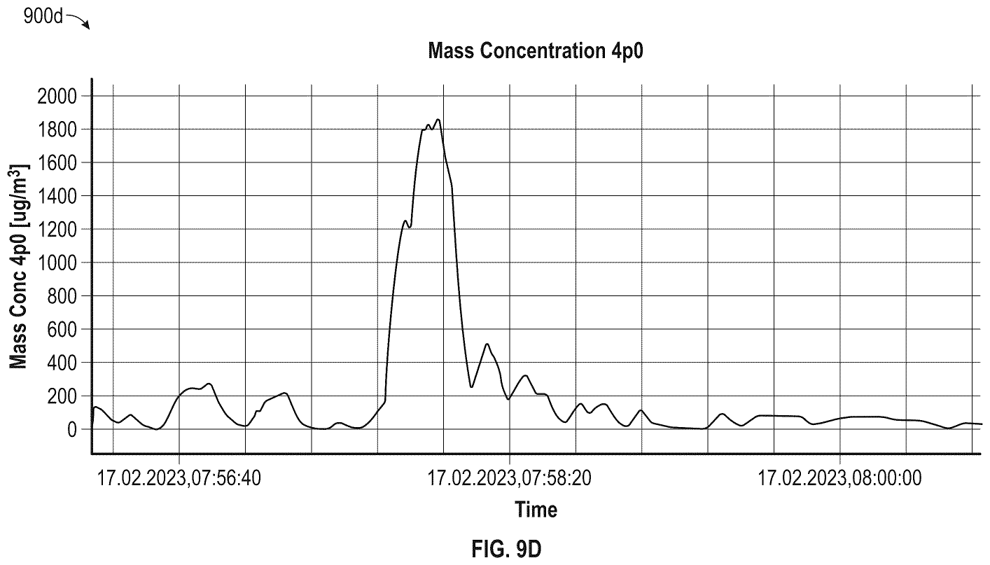

FIG. 2B is a block diagram of the sensing apparatus in accordance with another illustrative embodiment. The sensing apparatus 200b includes a housing 202 that houses a flow path 204 in fluid communication with an enclosable environment, such as enclosable environment 102. The flow path 204 has an inlet 206 that receives air, represented by arrow 208, from the enclosable environment 102 and an outlet 210 that expels air into the enclosable environment 102. A plurality of sensors 212a, 212b, and 212c (collectively, sensors 212) can be exposed to the air in the flow path 204 for generating raw sampling data that describes at least two characteristics of contaminants in the air. An example of sensing data that can be captured by the plurality of sensors 212 can include particle mass concentration (μg/cm3), humidity (% RH), temperature (° C.), and surface oxygen in the air, which can be used to determine the presence of VOCs and nitrogen oxides (NOx) in the air. Example values of actual sensing data are shown in Table 1 above, and also in FIGS. 8A-8D and FIGS. 9A-9D. As can be seen in FIGS. 8A-8D and FIGS. 9A-9D, the values for particle mass concentration can range from the detection threshold to about 6,500 μg/cm3.

Sensor 212a can be configured to detect particulate matter suspended in the air of the enclosable environment 102. The sensor 212a can use any currently existing or later developed sensing technology, including infrared detection, beta attenuation mass monitoring, and laser diffraction, as long as it is configurable for the detection of the contaminants at issue. In the non-limiting embodiment depicted in FIG. 2B, the sensor 212a is a particulate matter (PM) sensor that operates by light scattering. Particulate matter 104 in the air flowing through the flow path 204 interacts with light (represented by arrow 209) generated by a light source 201, e.g., a laser. The light is generated according to control signals provided by laser controller 207 based on instructions from a microcontroller 216. The light is scattered towards a photodetector 203, e.g., a photodiode. The photodetector 203 can generate a signal that can then be processed by amplifier and filter 205 before transmission to microcontroller 216 for determining particle count (in particles per cubic centimeter) and mass concentration (in micrograms per cubic meter). An example of data collected by sensor 212a can be found in FIGS. 8A-8D and FIGS. 9A-9D.

Sensor 212b can be configured to detect a relative humidity and temperature of the air 208 flowing through the flow path 204. The sensor 212b can use any currently existing or later developed sensing technology, including capacitive technologies, resistive technologies, and thermal technologies. The sensor 212b can generate one output signal for humidity (in % RH), and a second output signal for temperature (e.g., in ° C. or ° F.). In some embodiments, the humidity and temperature can be used to identify characteristic signatures of contaminants in the air, which can then be used to determine the source of the contaminants. In other embodiments, the humidity and/or temperature can be used to determine whether the humidity and/or temperature may be influencing the raw sampling data captured by the sensors 212.

Sensor 212c can be configured to detect an amount of surface oxygen in the air flowing through flow path 204, which can be used to determine the relative amount of VOCs. The sensor 212c can use any currently existing or later developed sensing technology. In a non-limiting embodiment, the sensor 212c is a metal oxide (MOX) sensor that includes a semiconductor material formed with metal oxides, like tin dioxide (SnO2). When heated, the metal oxide catalyzes a reaction with the molecules that form the contaminants 104 in the air, which changes the electrical resistance of the MOX sensor based on the amount of molecular oxygen present. For example, VOCs, which are byproducts of the combustion of tobacco products, reduce the resistance of the MOX sensor proportionately to the concentration of VOCs present in the air by consuming oxygen. In contrast, NOx provides more oxygen at the metal oxide interface and increases the resistance of the MOX sensor proportionately to the concentration of NOx present in the air. The presence and/or absence of VOCs and/or NOx can be used to determine a source of the contaminants in the air.

The air can be drawn through the flow path 204 by a fan 240 receiving control signals from a fan controller 242 that is in turn controlled by the microcontroller 216. Although the fan 240 is shown at the outlet end of the flow path 204, in another embodiment, the fan 240 can be disposed at the inlet end of the flow path 204, or somewhere between the two.

During operation, the microcontroller 216 determines whether the mass concentration of the particulate matter in the air flowing through the flow path 204 exceeds a detection threshold. An exemplary detection threshold 902 is shown in FIG. 9A. Mass concentration values that exceed the detection threshold are deemed to be contamination events that trigger the capture of raw sampling data and contextual data and the generation of data entries as shown in Table 1. The data entries can be stored in a local storage 222.

At least some of the contextual data included in the data entries can be captured by GPS device 220. For example, GPS device 220 can capture location data and also timestamp data. In embodiments where GPS device 220 is replaced by another type of locating device, the timestamp data can be captured by an embedded clock or timekeeping circuit (not shown).

Data can be transmitted from the sensing device 200b to a remote computing device, such as remote computing device 106, via a communications interface 213. The communications interface 213 can be any form of currently existing or later developed communications device configured to communicate over a network, such as network 114, or over near field communications links. The data that can be transmitted can include data stored in the one or more data entries 215 and/or sensing results in the event that the sensing apparatus 200b is tasked with determining the source of the contaminants in the enclosable environment 102. To reduce the likelihood that an occupant of the enclosable environment 102 will be notified of the presence of sensing apparatus 200b, the communications interface 213 can refrain from attempting to pair with a remote computing device until after a physical pairing request has been received. In a non-limiting embodiment, the physical pairing request is the change in state of a magnetic switch, e.g., a reed switch 236, induced by a magnetic key 1900.

FIG. 2C is another schematic diagram of a sensing apparatus for sampling air within an enclosable environment according to an illustrative embodiment. The sensing apparatus 200c includes a housing 202 that houses a flow path 204 in fluid communication with an enclosable environment, such as enclosable environment 102. The sensing apparatus 200c is similar to the sensing apparatus 200a in FIG. 2A, but depicts the LUX sensor 263, obstruction detector 262, and supercapacitor 227 as a part of the local power supply 226.

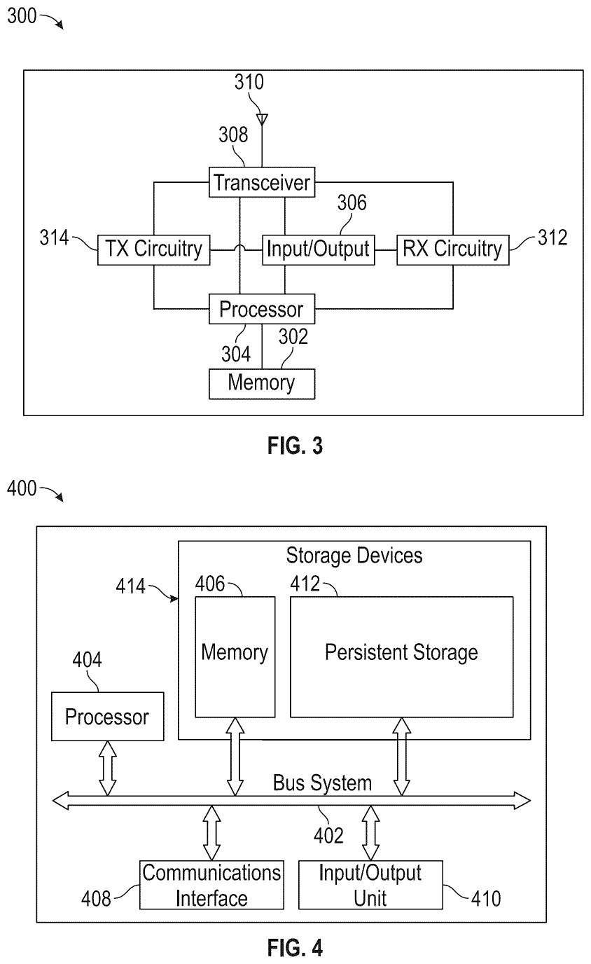

FIG. 3 is a block diagram of a non-limiting example of a mobile computing device for use in the detection and remediation of airborne contaminants according to an illustrative embodiment. Examples of mobile computing devices 300 can include cell phones, tablets, desktop computers, and the like. The mobile computing device 300 is provided for illustration only. The mobile computing devices 106 and 108 in FIG. 1 can have the same or similar configuration as mobile computing device 300 in FIG. 3.

Mobile computing device 300 includes memory 302 storing instructions that can be executed by processor 304 for controlling the operation of the mobile computing device 300. For example, the memory can store an operating system and one or more applications that can be executed by the processor 304. The memory 302 can include random access memory (RAM), Flash memory, and/or read-only memory (ROM).

I/O 306 is one or more input/output (I/O) devices of the mobile computing device 300. Examples of I/O devices include, but are not limited to, a microphone, a speaker, a camera, a touch screen, a keypad. I/O 306 enables a user to interact with the mobile computing device 300 to communicate with a client, i.e., via a phone call, text message, email, or videoconference. In some embodiments, I/O 306 also includes I/O interfaces that provide the mobile computing device 300 with communications paths with other devices, such as other client devices and peripherals.

The transceiver 308 provides a wireless communications capability with a network, such as network 114 in FIG. 1. Incoming signals are received by the transceiver 308 from the antenna 310 and processed by the receive (RX) circuitry 312, which processes the signal and transmits the processed signal to an I/O device, such as a speaker, if the processed signal is for voice data. The processed signal can also be transmitted to the processor 304 for further processing before presentation to a user on another I/O device, such as a screen, if the processed signal is for other forms of data, such as web browsing data. Outgoing signals transmitted by the transceiver 308 from the antenna 310 are received from transmit (TX) circuitry 314. The TX circuitry 314 can receive voice data from a microphone, or other forms of outgoing data, such as web data, e-mail, or application data, from the processor 304.

Although the mobile computing device 300 in FIG. 3 is depicted as a mobile phone, the mobile computing device 300 can be selected from any other conventional mobile computing devices such as tablets and laptop computers, or a suitably configured custom device. For example, the transceiver depicted in the mobile computing device 300 can be replaced by a network communications interface that can support wired or wireless communication over network 114.

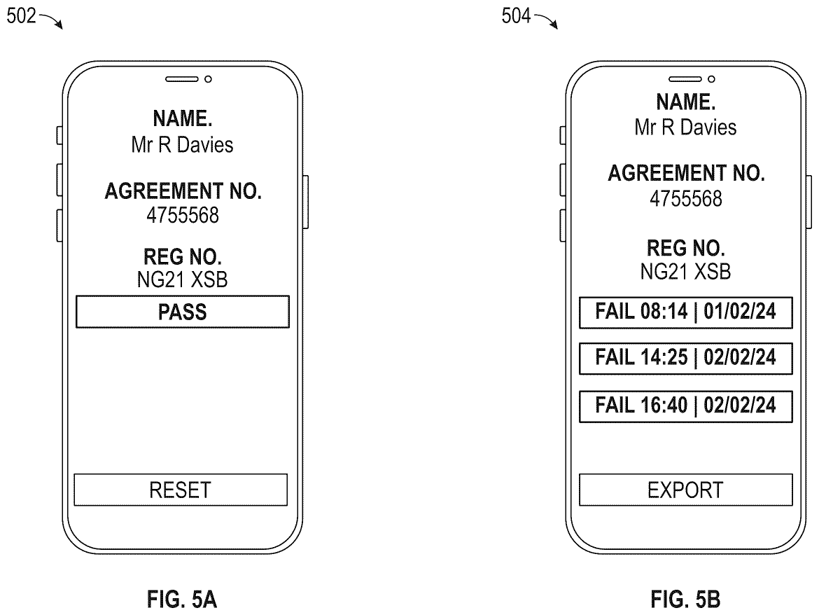

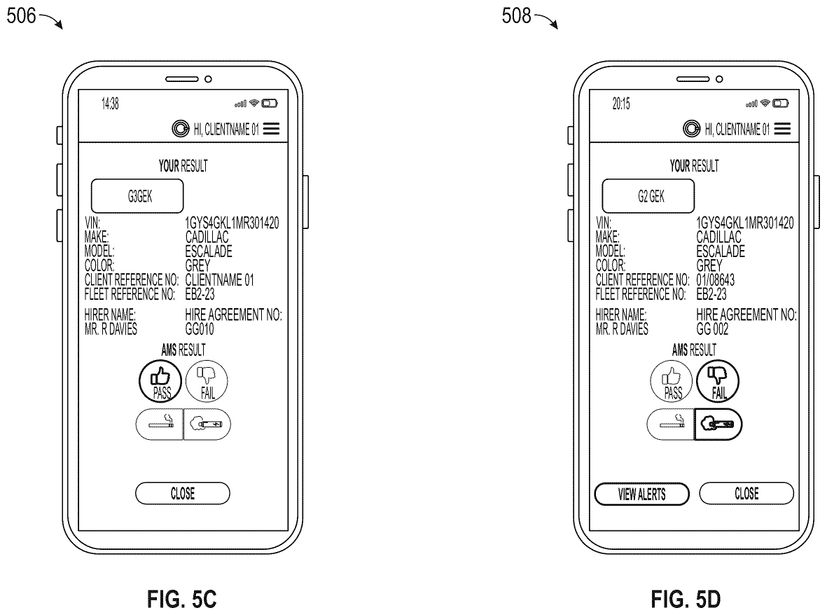

In an exemplary use case, a user of the mobile computing device 300 can be tasked with inspecting the enclosable environment 102 after a renter or occupant of the enclosable environment 102 has relinquished possession or control. The user can bring the mobile computing device 300 into proximity of the enclosable environment 102 and establish a communications link with the sensing apparatus 200 mounted within the enclosable environment 102. Regardless of whether the sensing results are generated by the sensing apparatus 200, the mobile computing device 300, or another remote computing device, such as server 400, the user of the mobile computing device 300 can be provided with a notification indicating whether one or more airborne contaminants 104 were detected within the enclosable environment. Exemplary notifications are shown in FIGS. 5A and 5B that follows. The notification can also include information that can associate the one or more airborne contaminants 104 with the enclosable environment 102 and optionally provide instructions on how to remediate the enclosable environment 102 to eliminate the presence of the one or more airborne contaminants 104.

FIG. 4 is a schematic diagram of a server for use in the detection and remediation of airborne contaminants according to an illustrative embodiment. The computing device 400 is provided for illustration only.

Server 400 includes a bus system 402 that supports communication between at least one processor 404, at least one storage device 414, at least one communications interface 408, and at least one input/output (I/O) unit 410.

The memory 406 and a persistent storage 412 are examples of storage devices 414, which represent any structure(s) capable of storing and facilitating retrieval of information (such as data, program code, and/or other suitable information on a temporary or permanent basis). Memory 406 may represent a random-access memory or any other suitable volatile or non-volatile storage device(s). The persistent storage 412 may contain one or more components or devices supporting longer-term storage of data, such as a read only memory, hard drive, Flash memory, or optical disc.

The processor 404 may execute instructions that may be loaded into memory 406. The processor 404 may include any suitable number(s) and type(s) of processors or other devices in any suitable arrangement. Example types of processors 404 include microprocessors, microcontrollers, digital signal processors, field programmable gate arrays, application specific integrated circuits, and discreet circuitry.

The communications interface 408 may support communications with other systems or devices. For example, the communications interface 408 could include a network interface card or a wireless transceiver facilitating communications over the network 102. The communications interface 408 may support communications through any suitable physical or wireless communication link(s).

The I/O unit 410 may allow for input and output of data. For example, the I/O unit 410 may provide a connection for user input through a keyboard, mouse, keypad, touchscreen, or other suitable input device. The I/O unit 410 may also send output to a display, printer, or other suitable output device.

In an exemplary use case, the server 400 can receive sensing data from a mobile computing device, such as mobile computing device 106, directly from a sensing apparatus 200, or from storage device 110, as described in the various examples above. Thus, the server 400 can analyze raw sampling data to generate sampling results, initiate remediation of an enclosable environment based on sampling results, determine compliance with rental agreements, and/or issue fines, if applicable.

FIGS. 5A, 5B, 5C, and 5D depict exemplary user interfaces provided on a mobile computing device for facilitating the detection and remediation of airborne contaminants in an enclosable environment according to an illustrative embodiment. The user interface 502 in FIG. 5A and the user interface 504 in FIG. 5B can be provided to an operator of a mobile computing device tasked with inspecting the enclosable environment 102. As an example, with particular reference to FIG. 1, the enclosable environment 102 is the interior of a vehicle that has been recently returned. The user of the mobile computing device 106 is tasked with inspecting the vehicle to ensure that the gas tank is full, and that the vehicle has not been damaged during use. When the user is within range of the communications interface 218 of the sensing apparatus 200, the sensing apparatus 200 and the mobile computing device 106 can begin transmitting data throughout system 100 according to one of the exemplary embodiments described in Examples 1-6.

The user can then be provided with user interface 502 if the sensing results indicate that no airborne contaminants were detected in the enclosable environment 102 during the rental period. The sensing results in FIG. 5A indicate an absence of airborne contaminants and take the form of a green PASS icon. The user interface 502 can also include additional information relevant to the rental of the vehicle, such as a unique identifier of the vehicle, e.g., the license plate, an identity of the renter, and a unique identifier of the rental agreement. Pressing the RESET icon can upload the sensing results to one or more network locations, such as storage device 110 or one or more remote computing devices and optionally clear the associated data from the mobile computing device 106.

If the sensing data provided by the sensing apparatus 200 indicates that one or more airborne contaminants 104 were detected in the enclosable environment 102 during the rental period, then the user of mobile computing device 106 can be provided with user interface 504. The sensing results in FIG. 5B indicate a presence of airborne contaminants and take the form of red FAIL icons. In the non-limiting example in FIG. 5B, the user interface 504 lists the instances during which the one or more airborne contaminants 104 were detected. Pressing one of the list elements can bring the user to another screen providing more contextual data, such as an identity of the airborne contaminant(s), date information, time information, location information, etc. In some embodiments, if the user of the mobile computing device 106 is also tasked with the remediation of the vehicle, then the user may also be provided with a remediation schedule indicating when the vehicle should be cleaned, as well as the approved methods of cleaning the vehicle. Pressing the EXPORT icon can upload the sensing results to one or more network locations, such as storage device 110 or one or more remote computing devices and optionally clear the associated data from the mobile computing device 106.

User interface 506 in FIG. 5C and user interface 508 in FIG. 5D are alternate embodiments of user interface 502 in FIG. 5A and the user interface 504 in FIG. 5B, respectively.

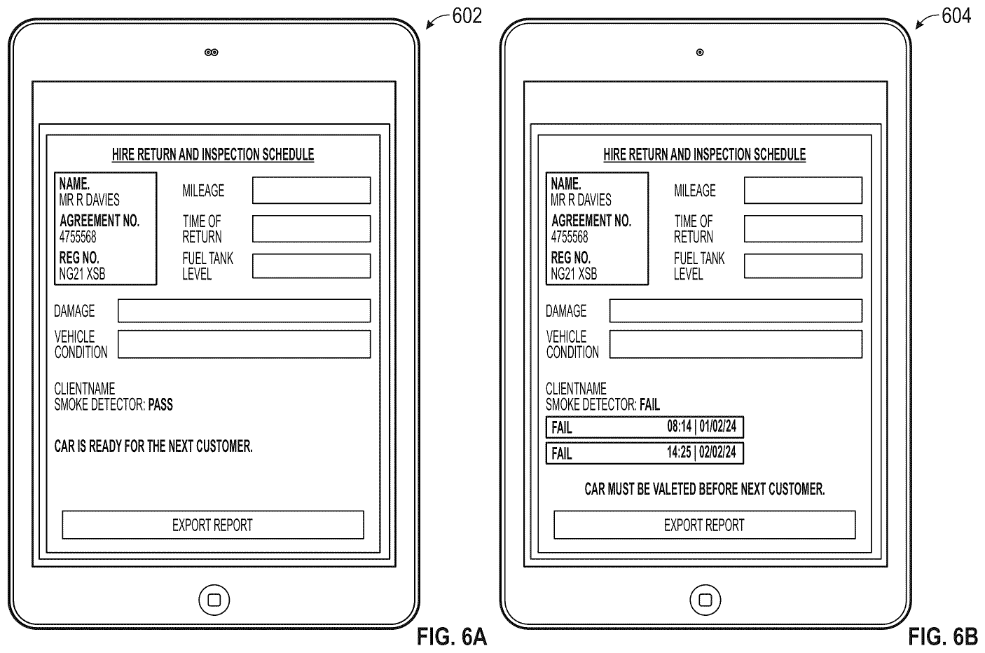

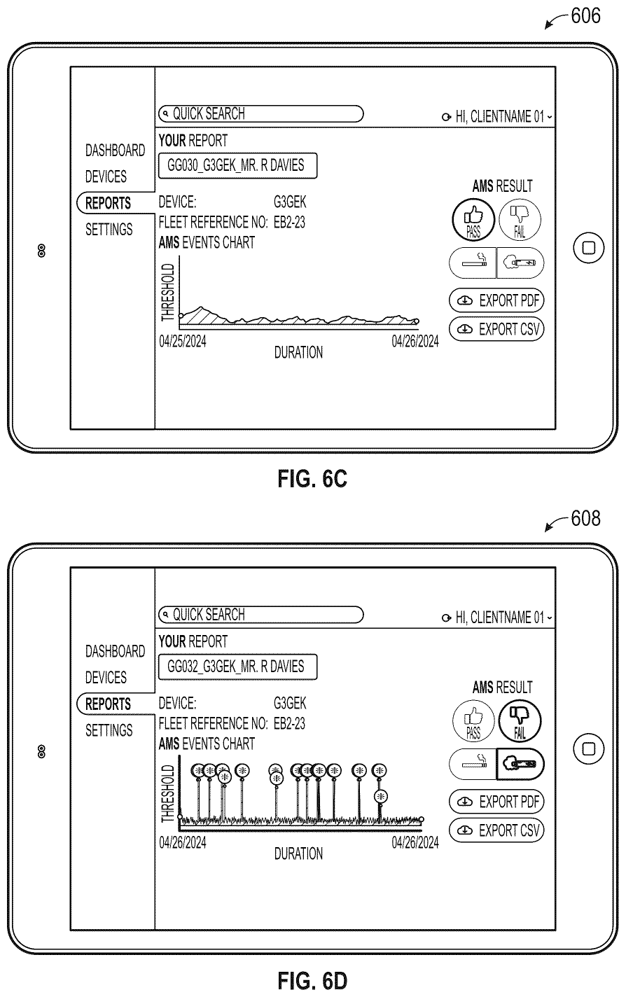

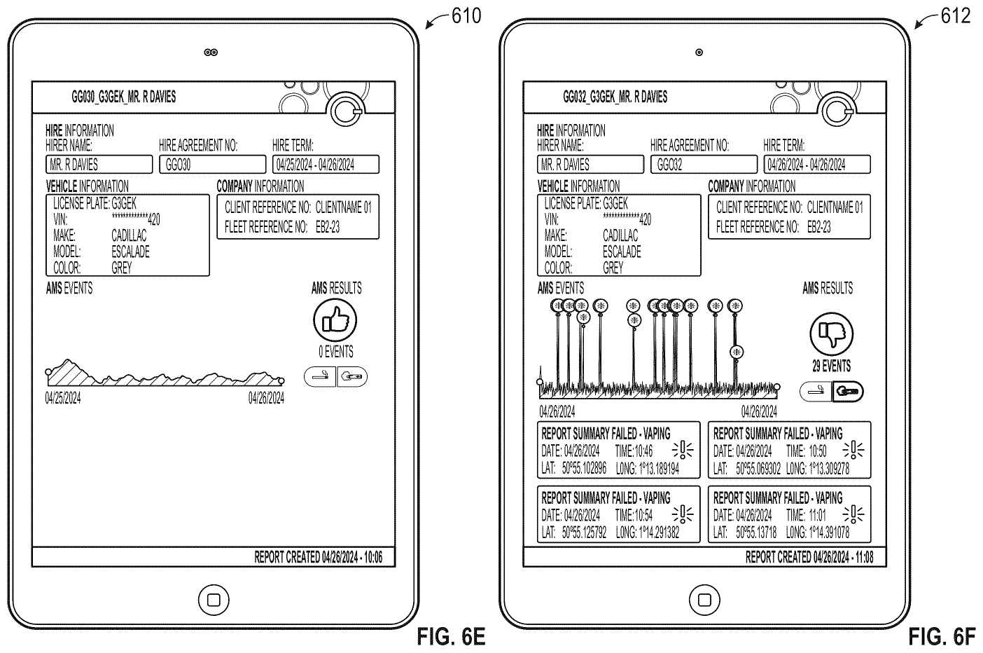

FIGS. 6A, 6B, 6C, 6D, 6E, and 6F depict exemplary user interfaces provided on a mobile computing device for facilitating the detection and remediation of airborne contaminants in an enclosable environment according to another illustrative embodiment. The user interface 602 in FIG. 6A and the user interface 604 in FIG. 6B can be provided to an operator of a computing device tasked with managing business records related to the use and occupation of the enclosable environment 102. As an example, with particular reference to FIG. 1, the enclosable environment 102 is the interior of a vehicle that has been recently returned. The user of the mobile computing device 108 is tasked with enforcing terms of a rental agreement. A portion of the electronic rental agreement is shown in user interfaces 602 and 604. In user interface 602, the rental agreement includes sampling results that indicate that no airborne contaminants were detected within the vehicle. In user interface 602, the rental agreement includes sampling results that indicate that one or more airborne contaminants were detected in the enclosable environment 102 on multiple occasions. As a result, the mobile computing device 108 can automatically initiate remediation of the vehicle, e.g., by scheduling a cleaning, updating the inventory with a projected availability of the vehicle, assess a fine, etc. In another embodiment, the operator of the mobile computing device 108 can initiate remediation of the vehicle by interacting with UI elements on the user interface 604.

User interface 606 in FIG. 6C and user interface 610 in FIG. 6E are alternate embodiments of the user interface 602 in FIG. 6A. User interface 608 in FIG. 6D and user interface 612 in FIG. 6F are alternate embodiments of the user interface 604 in FIG. 6B.

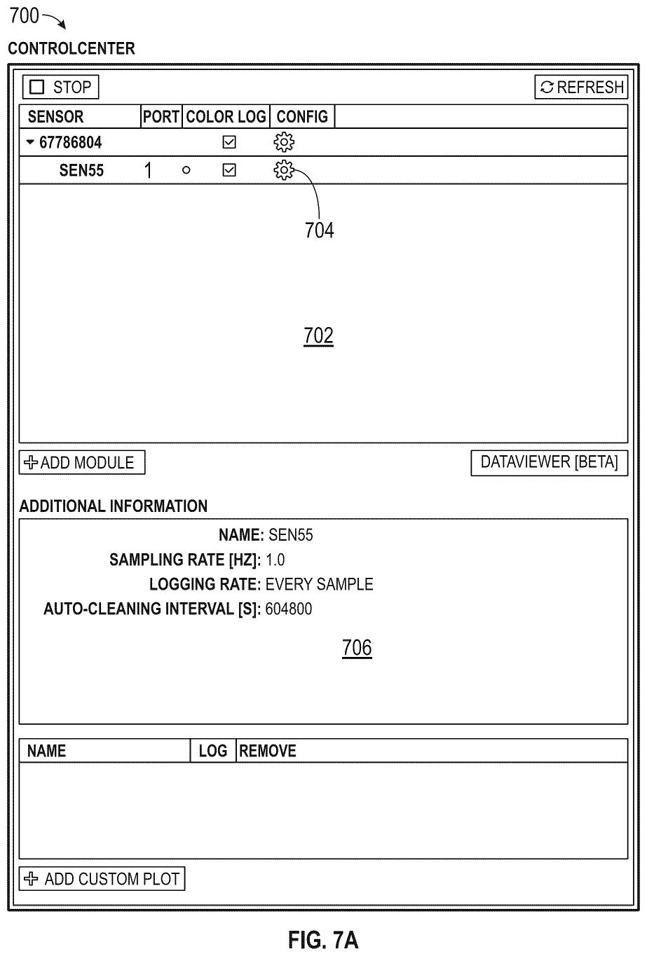

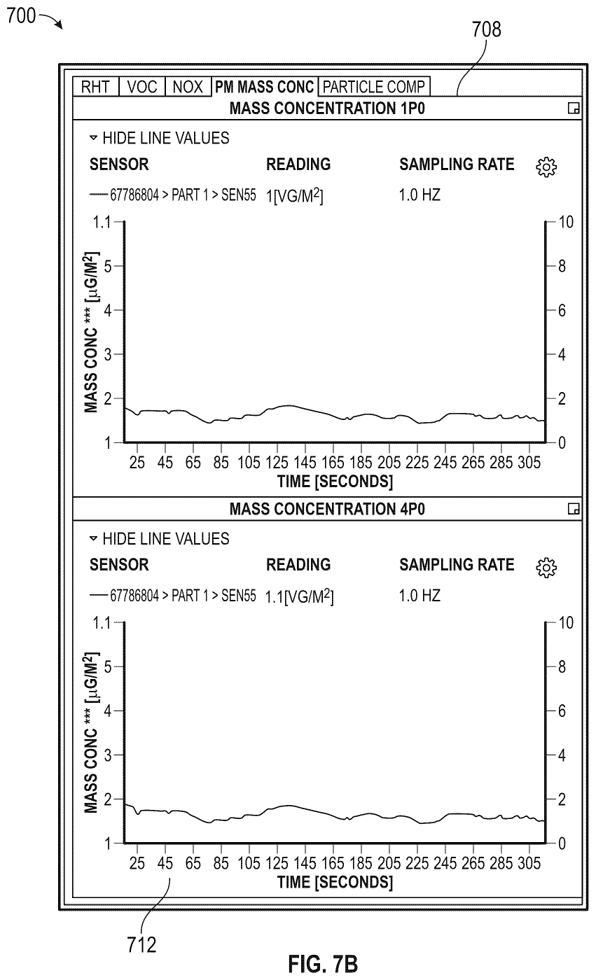

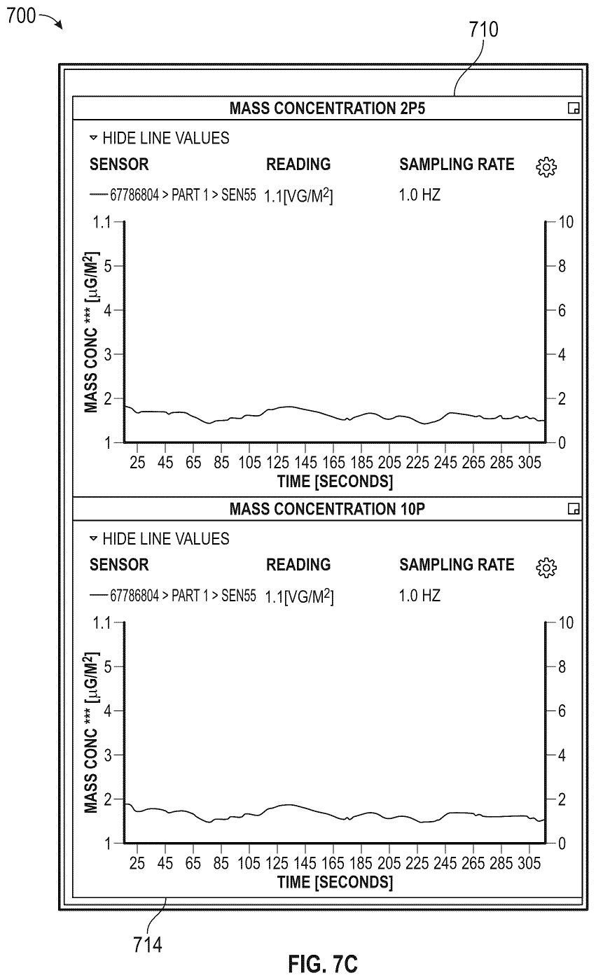

FIG. 7 depicts an exemplary user interface for viewing raw sampling data according to an illustrative embodiment. The user interface 700 includes a first windowed UI element 702 that lists the sensors associated with the enclosable environment. In this example, the enclosable environment has just one sensor, e.g., sensing apparatus 200 disposed within enclosable environment 102, as depicted in FIG. 1. The gear icon 704 can be selected to allow sampling settings to be selected. The selected sampling settings can be shown in the second windowed UI element 706.

Windowed UI elements 708, 710, 712, and 714 depict raw sampling data captured using different sampling metrics. In the illustrative example in FIG. 7, the sensing apparatus 200 identified in the first windowed UI element 702 uses beta attenuation mass monitoring for sampling air in an enclosable environment. Windowed UI element 708 shows particles with a particle mass of 1p0, i.e., particles between 0.3-1.0 μm, windowed UI element 710 shows particles with a particle mass of 2p5, i.e., particles between 1.1-2.5 μm, windowed UI element 712 shows particles with a particle mass of 4p0, i.e., particles between 2.6-4.0 μm, and windowed UI element 714 shows particles with a particle mass of 10p, i.e., particles between 4.1-10.0 μm.

In the event that the enclosable environment 102 is a rented space (e.g. a rented car or a room) and is being handed over to a renter/occupant, the sensor apparatus 200 can be used to sample the air within the enclosable environment 102 to determine a baseline of particulate matter. During the renter's use of the space, raw sampling data can then be collected and compared relative to the baseline to determine whether particulate matter in the air is attributable to actions of the renter/occupant. For example, a newly manufactured car may generate VOCs as the new interior continues its usual off-gassing. The baseline measurements, shown in the various windowed UI elements 708, 710, 712, and 714, can be used to account for the ambient particulate matter not attributable to the actions of the renter/occupant of the enclosable environment 102.

FIGS. 8A-8D and FIGS. 9A-9D are graphs of raw sampling data collected by a sensing apparatus 200 during a contamination event. In particular, FIGS. 8A-8D depict raw sampling data captured in an enclosable environment 102 over a two-hour period according to an illustrative embodiment, and FIGS. 9A-9D depict raw sampling data captured in an enclosable environment 102 over a four-minute period according to an illustrative embodiment.

The exemplary sensing apparatus 200 of this disclosure includes a particulate sensor 212a that can determine the mass concentration of particulates in micrograms per cubic meter to generate graphs depicting the mass concentration of particulates versus time. FIGS. 8A and 9A are graphs that depict the mass concentration of particulates that are between 4.1-10 μm in size; FIGS. 8B and 9B are graphs that depict the mass concentration of particulates that are between 0.3-1 in size; FIGS. 8C and 9C are graphs that depict the mass concentration of particulates that are between 1.1-2.5 μm in size; and FIGS. 8D and 9D are graphs that depict the mass concentration of particulates that are between 2.6-4.0 μm in size. Graphs 800a, 800b, 800c, and 800d depict high plume contamination events with larger mass concentrations and low plume contamination events with smaller mass concentrations. Exemplary high plume contamination events correspond to peaks 802 and 806, and low plume contamination events correspond to peaks 804 and 808. The highest peak in each of graphs 900a, 900b, 900c, and 900d corresponds to a low plume contamination event. The dashed line 902 in FIG. 9A is an exemplary detection threshold of about 300 μg/m3, which was selected to eliminate or at least reduce the likelihood of false positives that may arise due to ambient conditions.

As previously mentioned, once a contamination event is detected with reference to raw sampling data that describes a first characteristic of the contaminants in the air, a data entry can be generated that captures raw sampling data and contextual data. With particular reference to the graphs shown in FIGS. 8A-8D and FIGS. 9A-9D, the first characteristic is a mass concentration of particulates in the air, and the detection threshold is 300 μg/m3. A plurality of contamination events is detected based on each peak that exceeds the 300 μg/m3 detection threshold, and for each contamination event a data entry is created with the raw sampling data and contextual data as shown in Table 1. The raw sampling data included data describing one or more second characteristics of the contaminants in the air (e.g., VOC, NOx, temperature, and/or humidity) can be used to determine the source of the contaminants. In a non-limiting embodiment, the magnitude of the VOCs in the air indicates that the source of the contaminants is a tobacco product and not a vaping product, which does not generate VOCs, or generates negligible or undetectable levels of VOCs. In a similar way, other sources of contaminants can be determined based on a unique signature of the raw sampling data.

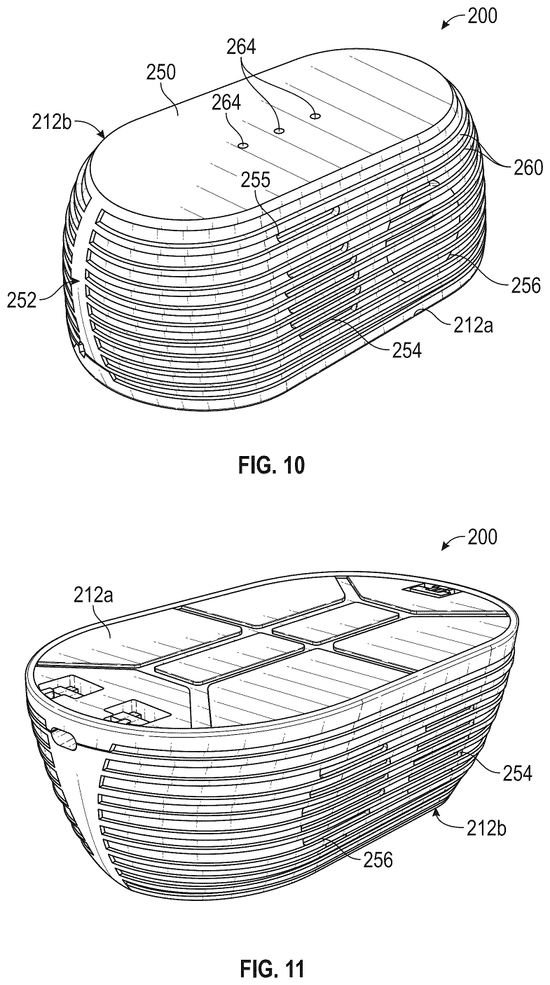

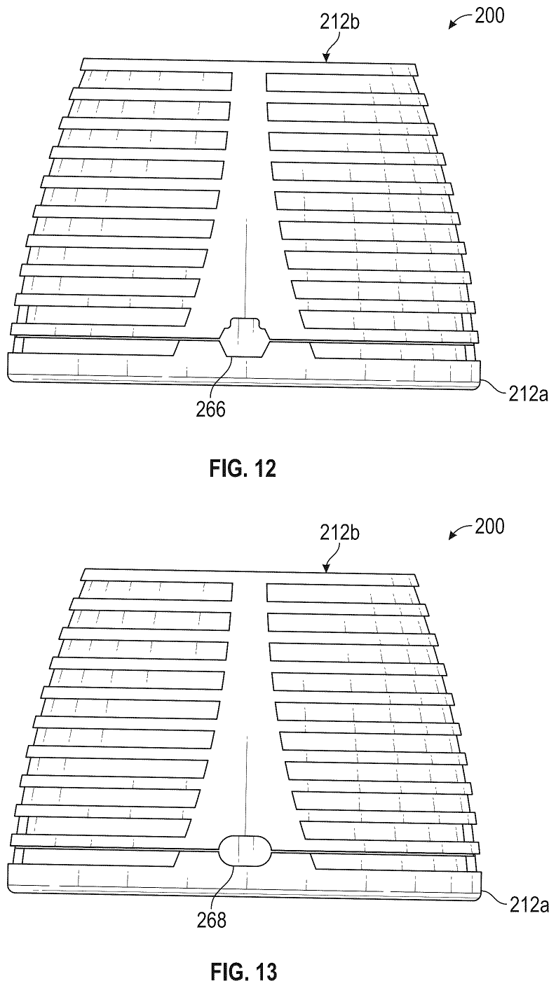

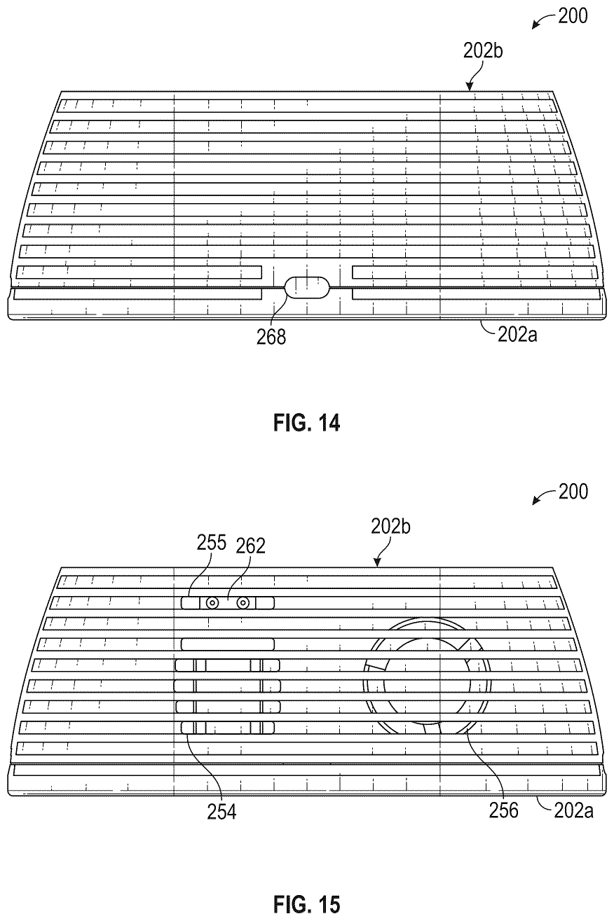

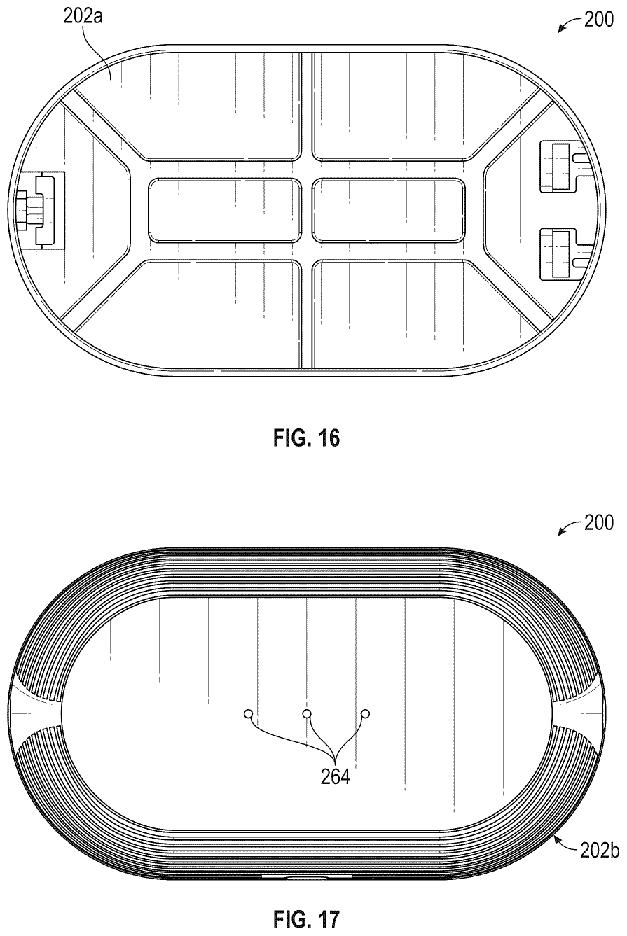

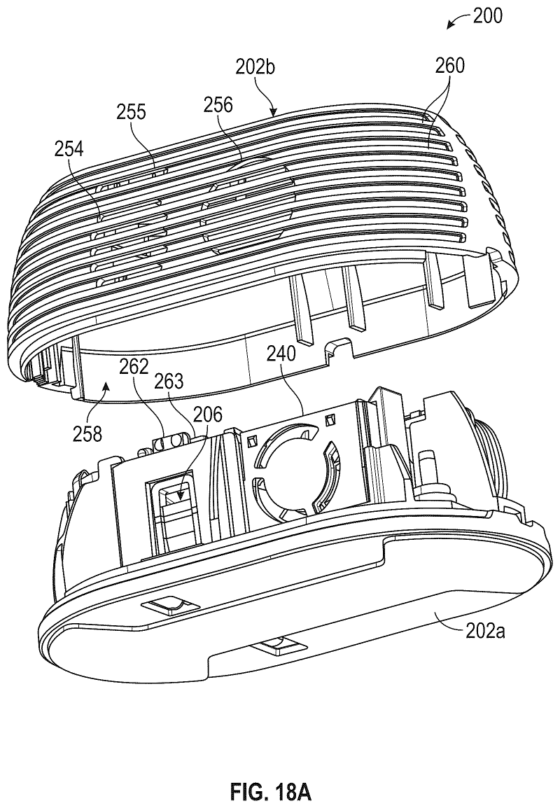

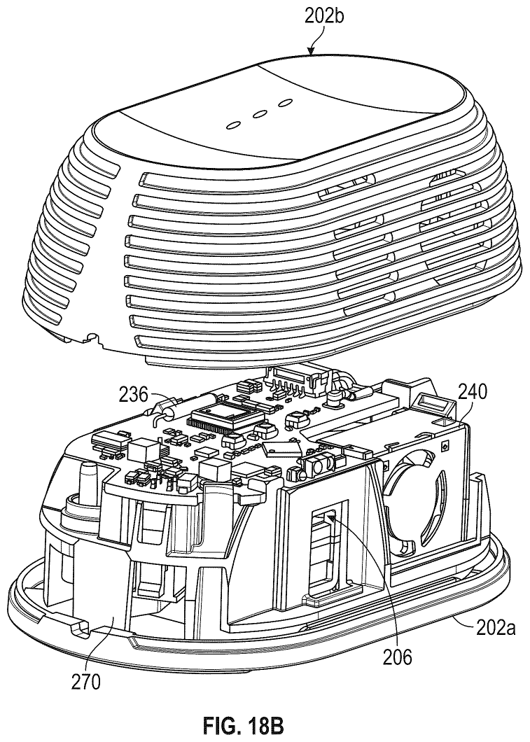

FIGS. 10-18 depict various views of the sensing apparatus 200 according to an illustrative embodiment. To facilitate the discussion of the sensing apparatus 200 in this disclosure, the base 202a of the sensing apparatus 200 will be referred to as the “bottom” and the surface of the housing cover 202b opposite the base 202a will be referred to as the “top”. Thus, FIG. 10 is a top perspective view of the sensing apparatus 200; FIG. 11 is a bottom perspective view of the sensing apparatus 200; FIGS. 12 and 13 are elevation views of the sensing apparatus 200 from each of the two ends; FIGS. 14 and 15 are side elevation views of the sensing apparatus 200; FIG. 16 depicts a bottom view of the sensing apparatus 200; and FIG. 17 depicts a top view of the sensing apparatus 200. FIGS. 18A and 18B are perspective views of the sensing apparatus 200, but with the housing cover disengaged from its base.

The sensing apparatus 200 is formed from a housing 202 that defines an enclosable cavity 258, shown in FIG. 18A, which houses the components of the sensing apparatus 200. The depicted exemplary housing 202 is formed from a base 202a and a housing cover 202b. The housing cover 202b includes a top surface 250 that is connected at its periphery to a side wall 252. Two apertures are formed in the side wall 252 of the housing cover 202b, a first aperture 254 that coincides with the inlet 206 of the flow path 204 and a second aperture 256 that coincides with the outlet 210 of the flow path 204 when the housing cover 202b is coupled to the base 202a. A third aperture 255 can be formed in the side wall 252 that allows an obstruction sensor 262 housed inside the housing 202, shown in more detail in FIG. 18A, to detect a presence of an obstruction.

The side wall 252 of the housing cover 202b imparts a geometry to the sensing apparatus 200 that is difficult to grasp when the base 202a is mounted within an enclosable environment 102, which can prevent or at least dissuade an occupant from attempting to remove the sensing apparatus 200. In particular, the side wall 252 has a transverse cross-sectional area (i.e., parallel to the base 202a) that is largest at the base and tapers to its narrowest at the top surface 250. The curved shape of the housing cover 202b also makes it more difficult to grasp.

In addition, the sensing apparatus 200 includes a set of ridges 260 projecting out of the side wall 252 and spanning the first aperture 254 and the second aperture 256 to make it more difficult for a malicious actor to obstruct the apertures 254 and 256. If the sidewall were straight and lacked ridges, then air flow through the flow path 204 can be easily obstructed by a barrier, e.g., tape or other object, placed in front of the apertures 254 and 256. The set of ridges 260 prevents the barrier from sealing against the apertures 256 and 258 and permits the ingress of air through the channels between each of the ridges 260. Additionally, the curvature in the sidewall 252 provides the sensing apparatus 200 a shape that is more difficult to accommodate when attempting to obstruct the apertures 254 and 256.

A fan 240 is secured within the housing 202 and positioned to coincide with the outlet 210 of the flow path 204 and also the aperture 258. Although not shown in FIGS. 10-18B, a plurality of sensors 212 are exposed to the air flowing through the flow path 204. The flow path 204 and the plurality of sensors 212 are depicted in the exemplary block diagram in FIG. 2B.

The sensing apparatus 200 can include an obstruction detector 262 mounted within the housing 202 and oriented to detect an obstruction of the inlet 206. Non-limiting examples of the obstruction detector 262 include an infrared detector, an ultrasonic detector, and a photodetector. The obstruction detector is configured to detect a presence of an obstruction over the inlet 206 which is intended to block the flow of air through the flow path 204. In some embodiments, the detection of an obstruction can trigger an audio driver 232 and speakers 234 to generate an audible alarm.

The sensing apparatus 200 can include a set of indicator lights 264 for communicating information to a user. Examples of the information can include an idle state, an advertising state in which the sensor apparatus 200 is looking to pair with a remote computing device, a paired state, and a data transmission state. The information can be conveyed by changing a color of the set of indicator lights 264 or by changing a pattern of illumination. A LUX sensor 263 is also positioned proximate to the third aperture 255 to detect light in the enclosable environment 102, which can be used to adjust the intensity of the set of indicator lights 264 based on ambient conditions. For example, the indicator lights 264 can be adjusted to a lower intensity at night to prevent distractions or reduce the likelihood of discovery by occupants of the enclosable environment 102.

To prevent unauthorized access to the internal components of the sensing apparatus 200, the housing cover 202b can be secured to the base 202a by a locking device accessible via a keyhole aperture 266. In this illustrative embodiment, the locking device is a flexible latch 270, depicted in FIG. 18B, which can be engaged by a magnetic key 1900 having a shaft 1904 that is keyed to fit into the keyhole aperture 266 to depress the flexible latch 270. Once depressed, the flexible latch 270 permits separation of the housing cover 202b from the base 202a. The sensing apparatus 200 can also include additional apertures 268 sized to allow the positive and negative wires of a power cable to pass through the housing 202.

With reference to FIG. 18A, it can be seen attachment of the housing cover 202b to the base 202a causes the first aperture 254 to align with the inlet 206 and the second aperture 256 to align with outlet 210. Likewise, aperture 255 aligns with the obstruction sensor 262, which is shown adjacent to the inlet 206 of the flow path 204 so that any attempt at covering up the first aperture 254 would cause the obstruction sensor 262 to indicate the presence of an obstruction.

With reference to FIG. 18B, the reed switch 236 is preferably mounted within the housing 202 at a location that allows the reed switch 236 to magnetically interface with a magnet to cause the reed switch 236 to change its state, which signals to the microcontroller 216 that a pairing function has been requested. In the illustrative embodiment in this disclosure, the reed switch 236 is mounted on a circuit board adjacent to the housing cover 202b, e.g., the top surface 250 of the housing cover 202b, so that a magnet pressed against the housing cover 202b can cause the reed switch 236 to change its state.

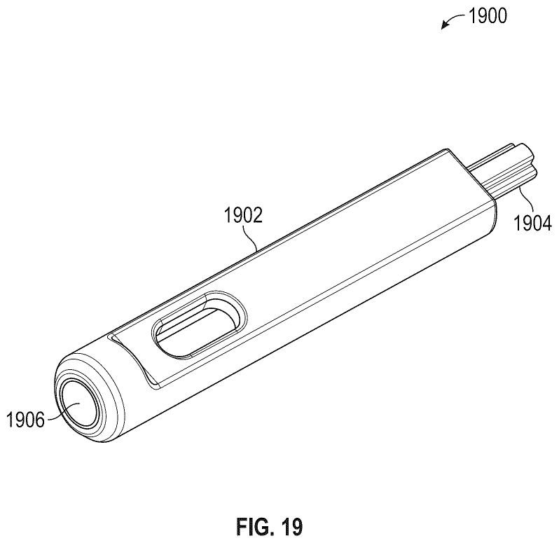

FIG. 19 is a perspective view of a magnetic key 1900 for use with the sensing apparatus 200 according to an illustrative embodiment. The magnetic key 1900 is shown having a generally elongated body 1902 with a shaft 1904 keyed to fit into the keyhole aperture 266. Insertion of the shaft 1904 into the keyhole aperture 266 causes the housing cover 202b to detach from the base 202a to expose the internal components of the sensing apparatus 200. The magnetic key 1900 also includes a magnet 1906 that can magnetically interface with the reed switch 236 for initiating a pairing function of the sensing apparatus 200 with a remote computing device, such as remote computing device 106 in FIG. 1. By preventing the sensing apparatus 200 from initiating a pairing function until after an exposure to a magnetic field, occupants of the enclosable environment are less likely to discover the existence of the sensing apparatus 200 and attempt to circumvent the detection capabilities of the sensing apparatus 200.

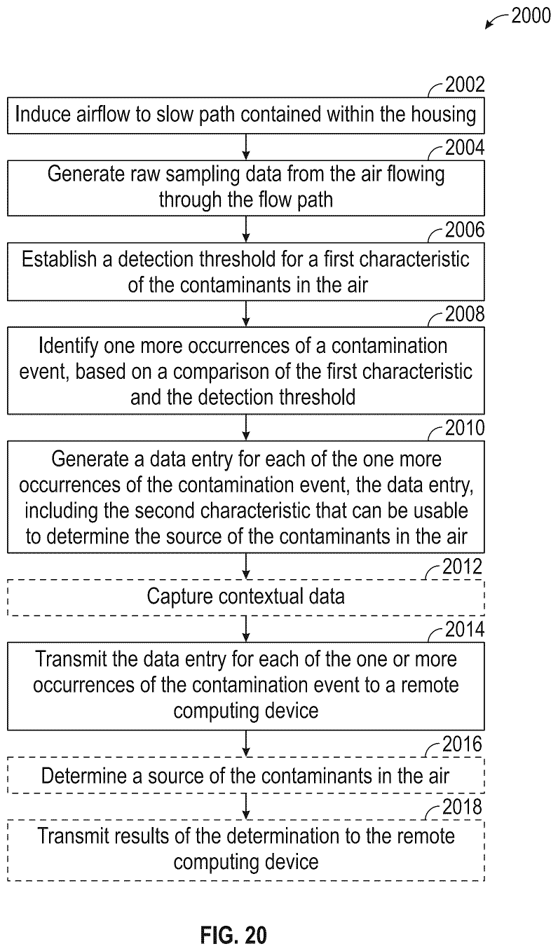

FIG. 20 is an exemplary embodiment of a flowchart of a process for determining a source of contaminants in air. The steps of flowchart 2000 can be carried out in a sensing apparatus, such as sensing apparatus 200.

Flowchart 2000 begins at step 2002 by inducing air flow through a flow path contained within a housing. In some embodiments, inducing the air flow can include powering a fan 240 disposed within the flow path 204. Additionally, inducing the air flow through the flow path 204 can include conveying the air across a plurality of sensors 212 configured to generate the raw sampling data. Further the step of inducing the flow of air can also include monitoring for an obstruction of the first aperture 254 coinciding with the inlet 206 of the flow path 204 and generating an alert in response to detecting the obstruction.

In step 2004, raw sampling data is generated from the air flowing through the flow path. The raw sampling data, which can be generated by the sensors 212, describes at least two characteristics of contaminants in the air. The first characteristic can be used to determine whether a contamination event has occurred, and the second characteristic can be used to determine a source of the contaminants.

In step 2006, a detection threshold is established for the first characteristic of the contaminants in the air. The detection threshold is used to eliminate or at least reduce the likelihood of false positive readings. The detection threshold can be established by receiving a predetermined value based on historical testing. In some instances, environmental conditions, e.g., higher levels of ambient pollutants or presence of smoke from nearby wildfires, may necessitate an increase detection threshold. In those instances, the detection threshold can be established by receiving periodically updated values for the detection threshold. In a non-limiting embodiment, the first characteristic can be a mass concentration of the contaminants in the air, and the detection threshold is a mass concentration between 50-300 μg/m³, or more specifically a mass concentration between 75-200 μg/m³. In a particular embodiment, the detection threshold is a mass concentration that is about 100 μg/m³. In these embodiments, the prior step of generating the raw sampling data further comprises detecting the mass concentration of the contaminants in the air.

In step 2008, one or more occurrences of a contamination event is identified based on a comparison of the first characteristic and the detection threshold. In the embodiment where the first characteristic is a mass concentration of contaminants in the air and the threshold is 100 μg/m³, a contamination event is identified when an air sample exceeds 100 μg/m³. For example, with reference to FIGS. 8A-8D, peaks 802, 804, 806, and 808 correspond to some of the contamination events detected by the sensor 200. Peaks 802 and 806 correspond to high plume contamination events that produce larger mass concentration of contaminants in the air. Peaks 804 and 808 correspond to low plume contamination events that produce relatively smaller mass concentrations of contaminants in the air. Notably, each of the contamination events 802, 804, 806, and 808 exceed the threshold of 100 μg/m³.

In step 2010, for each of the one or more occurrences of the contamination event, a data entry is generated that includes the second characteristic. The second characteristic is usable to determine the source of the contaminants in the air. The second characteristic can include at least one of a relative humidity of the air and an amount of surface oxygen in the air. In these embodiments, the step of generating the raw sampling data can include detecting at least one of the relative humidity of the air flowing through the flow path and the amount of surface oxygen in the air. In a particular embodiment, the second characteristic is presence of VOCs, as determined by the amount of surface oxygen in the air.

The data entry can also include data related to the first characteristic as well as contextual data captured in optional step 2012. Examples of contextual data can include location data that can be included in each data entry for the one or more occurrences of the contamination event. The location data can be generated by cell tower triangulation, proximity to WIFI-enabled devices with known locations, and/or Global Positioning System (GPS) modules. The contextual data can also include a timestamp. In some embodiments, the capturing of contextual data occurs only when the one or more occurrences of the contamination event occurs to reduce the amount of data stored and transmitted by the sensing apparatus 200.

Flowchart 2000 can also include transmitting the data entry for each of the one or more occurrences of the contamination event to a remote computing device in step 2014. In some embodiments, the transmitting step can include initiating pairing of a communications interface of the sensing apparatus with a remote computing device in response to detecting a change in state of a magnetic switch. The magnetic switch can be a reed switch that can detect a presence of a magnetic field by changing its state. The magnetic field can be supplied by the magnetic key 1900 depicted in FIG. 19. By preventing the sensing apparatus 200 from initiating a pairing function until after an exposure to a magnetic field, occupants of the enclosable environment are less likely to discover the existence of the sensing apparatus 200 and attempt to circumvent the detection capabilities of the sensing apparatus 200.

The source of the contaminants in the air can be determined based on the second characteristic. If the source of the contaminants in the air is determined by a remote computing device, such as remote computing device 106, or a computing device communicatively coupled to the computing device over a network, such as computing device 400, then flowchart 2000 can end at step 2014. If the source of the contaminants is determined by the sensing apparatus 200, then flowchart 2000 can proceed from step 2014 to step 2016 where the sensing apparatus 200 determines the source of the contaminants according to the discussion accompanying FIG. 8. The results of the determination can be transmitted to a remote computing device, e.g., remote computing device 106, in step 2018. The remote computing device can use the data entry for each of the one or more occurrences of the contamination event and the results of the determination to initiate remediation of the enclosable environment, or forward the data received from the sensing apparatus 200 to another computing device for initiating remediation.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. An apparatus for use in determining a source of contaminants in air, the apparatus comprising:
a plurality of sensors configured to generate raw sampling data of the air, wherein the raw sampling data describes at least two characteristic of the contaminants in the air;
memory storing instructions;
a processor communicatively coupled to the set of sensors and the memory, the processor configured to execute the instructions to cause the apparatus to:
identify one or more occurrences of a contamination event based on a comparison of a first characteristic and a detection threshold for the first characteristic of the contaminants in the air; and
for each of the one or more occurrences of the contamination event, generate a data entry including a second characteristic, wherein the second characteristic is usable to determine the source of the contaminants in the air.

2. The apparatus of claim 1, wherein:
the first characteristic is a mass concentration of the contaminants in the air, and
the plurality of sensors includes at least a particle mass sensor configured to detect the mass concentration of the contaminants in the air.

3. The apparatus of claim 2, wherein the detection threshold is a mass concentration between 50-300 μg/m³.

4. The apparatus of claim 1, wherein:
the second characteristic includes at least one of a relative humidity of the air and an amount of surface oxygen in the air, and
the plurality of sensors includes at least one of a relative humidity sensor and a metal oxide sensor.

5. The apparatus of claim 1, further comprising a locating device coupled to the processor, wherein:
the locating device is configured to generate location data, and
the location data is included as contextual data in each data entry for the one or more occurrences of the contamination event.

6. The apparatus of claim 5, wherein the locating device is a Global Positioning System (GPS) module generating the location data and a timestamp, and the contextual data includes the location data and the timestamp.

7. The apparatus of claim 1, further comprising a communications interface coupled to the processor, wherein the processor is configured to execute the instructions to transmit the data entry for each of the one or more occurrences of the contamination event to a remote computing device via the communications interface for determining the source of the contaminants in air.

8. The apparatus of claim 7, wherein the processor is configured to execute the instructions to:
to determine the source of the contaminants in the air based on the second characteristic, and
transmit a result of the determination to the remote computing device.

9. The apparatus of claim 7, further comprising a magnetic switch communicatively coupled to the processor, wherein the processor is configured to execute the instructions to initiate pairing of the communications interface with the remote computing device in response to detecting a change in state of the magnetic switch.

10. The apparatus of claim 1, further comprising:
a housing defining an enclosable cavity, the housing including a first aperture and a second aperture;
a flow path disposed within the housing, the flow path having an inlet coinciding with the first aperture and an outlet coinciding with the second aperture;
a fan configured to induce air flow through the flow path; and
the plurality of sensors exposed to the air flow through the flow path.

11. The apparatus of claim 10, wherein the housing comprises at least one curved sidewall and a set of ridges projecting out of the curved sidewall and spanning the first aperture and the second aperture.

12. The apparatus of claim 1, further comprising:
an obstruction detector mounted within the housing and oriented to detect an obstruction of the inlet.

13. The apparatus of claim 1, wherein the obstruction detector is one of an infrared detector, an ultrasonic detector, and a photodetector.

14. A method for determining a source of contaminants in air, the method comprising:
inducing air flow through a flow path contained within a housing;
generating raw sampling data from the air flowing through the flow path, wherein the raw sampling data describes at least two characteristics of contaminants in the air;
establishing a detection threshold for a first characteristic of the contaminants in the air;
identifying one or more occurrences of a contamination event based on a comparison of the first characteristic and the detection threshold;
for each of the one or more occurrences of the contamination event, generating a data entry including the second characteristic, wherein the second characteristic is usable to determine the source of the contaminants in the air.

15. The method of claim 14, wherein:
the first characteristic is a mass concentration of the contaminants in the air, and
generating the raw sampling data further comprises detecting the mass concentration of the contaminants in the air.

16. The method of claim 15, wherein the detection threshold is a mass concentration between 50-300 μg/m³.

17. The method of claim 14, wherein:
the second characteristic includes at least one of a relative humidity of the air and an amount of surface oxygen in the air, and
generating the raw sampling data further comprises detecting at least one of the relative humidity of the air flowing through the flow path and the amount of surface oxygen in the air;

18. The method of claim 14, further comprising:
generating location data, wherein the location data is included as contextual data in each data entry for the one or more occurrences of the contamination event.

19. The method of claim 18, wherein:
the location data is generated by a Global Positioning System (GPS) module, and
the contextual data includes the location data and a timestamp provided by the GPS module.

20. The method of claim 14, further comprising:
transmitting the data entry for each of the one or more occurrences of the contamination event to a remote computing device.

21. The method of claim 20, further comprising:
determining the source of the contaminants in the air based on the second characteristic, and
transmitting a result of the determination to the remote computing device.

22. The method of claim 20, further comprising:
initiating pairing of the communications interface with remote computing device in response to detecting a change in state of a magnetic switch.

23. The method of claim 14, wherein inducing the air flow through the flow path further comprises conveying the air flow across a plurality of sensors configured to generate the raw sampling data.

24. The method of claim 14, wherein inducing the air further comprises powering a fan disposed within the flow path.

25. The method of claim 14, wherein inducing the flow of air further comprises:
monitoring for an obstruction of the inlet; and
generating an alert in response to detecting the obstruction.

26. A system for use in determining a source of contaminants in air, the system comprising:
an apparatus mounted within an at least partially enclosed environment and configured to:
generate raw sampling data of the air, wherein the raw sampling data describes at least two characteristics of the contaminants in the air,
identify one or more occurrences of a contamination event based on a comparison of a first characteristic and a detection threshold for the first characteristic of the contaminants in the air, and
for each of the one or more occurrences of the contamination event, generate a data entry including a second characteristic of the contaminants in the air; and
a remote computing device located externally from the at least partially enclosed environment, the remote computing device configured to receive the data entry for each of the one or more occurrences of the contamination event, wherein one of the apparatus or the remote computing device determines the source of the contaminants in the air from the second characteristic.

27. The system of claim 26, wherein:
the first characteristic is a mass concentration of the contaminants in the air, and
the apparatus includes a particle mass sensor configured to detect the mass concentration of the contaminants in the air.

28. The system of claim 27, wherein the detection threshold is a mass concentration between 50-300 μg/m³.

29. The system of claim 26, wherein:
the second characteristic includes at least one of a relative humidity of the air and an amount of surface oxygen in the air, and
the apparatus includes at least one of a relative humidity sensor and a metal oxide sensor.

30. The system of claim 26, wherein:
the apparatus includes a locating device configured to generate location data, and
the location data is included as contextual data in each data entry for the one or more occurrences of the contamination event.

31. The system of claim 30, wherein the locating device is a Global Positioning System (GPS) module generating the location data and a timestamp, and the contextual data includes the location data and the timestamp.

32. The system of claim 26, wherein the apparatus further comprises a communications interface configured to transmit the data entry for each of the one or more occurrences of the contamination event to the remote computing device via the communications interface.

33. The system of claim 32, wherein the apparatus further comprises a magnetic switch, and wherein the communications interface is configured to pair with the remote computing device in response to detecting a change in state of the magnetic switch.

34. The system of claim 26, wherein the apparatus further comprises:
a plurality of sensors disposed within a flow path and configured to generate the raw sampling data; and
a fan configured to induce a flow of air through the flow path across the plurality of sensors, wherein.

35. The system of claim 34, wherein the apparatus further comprises a housing that includes at least one curved sidewall and a set of ridges projecting out of the curved sidewall and spanning an inlet and an outlet of the flow path.

36. The system of claim 35, wherein the apparatus further comprises an obstruction detector mounted within the housing and oriented to detect an obstruction of the inlet.

37. The system of claim 36, wherein the obstruction detector is one of an infrared detector, an ultrasonic detector, and a photodetector.
